(19) United States
(12) Patent Application Publication
(10) Pub. No.: US 2025/0346968 A1
(43) Pub. Date: Nov. 13, 2025

(54) METHODS FOR DETECTING AND TREATING HEAD AND NECK CANCER

(71) Applicant: THE UNIVERSITY OF CHICAGO, Chicago, IL (US)

(72) Inventors: Evgeny G. Izumchenko, Chicago, IL (US); Nishant Agrawal, Chicago, IL (US); Vasudha Mishra, Chicago, IL (US)

(21) Appl. No.: 19/207,064

(22) Filed: May 13, 2025

Related U.S. Application Data
(60) Provisional application No. 63/646,611, filed on May 13, 2024.

(51) Int. Cl. C12Q 1/70; C12Q 1/6886

(52) U.S. Cl. CPC C12Q 1/70; C12Q 1/6886; C12Q 2600/112

(57) ABSTRACT

Aspects herein include methods, kits, and systems for evaluating a subject comprising detecting genetic mutation(s) in the DNA sequence of one or more oral cavity squamous cell carcinoma (OCSCC) biomarker(s) in a biological sample from the subject comprising DNA, wherein the OCSCC biomarker(s) comprise TP53, CDKN2A, FAT1, CASP8, NOTCH1, PIK3CA, and/or HRAS along with detecting or determining an HPV infection.

### FIELD OF THE INVENTION

This invention relates to the detection and treatment of head and neck cancers including oral cavity squamous cell carcinoma (OCSCC) and oropharyngyl squamous cell carcinoma (OPSCC).

### BACKGROUND

Head and neck cancer (HNC) is a frequently diagnosed cancer, accounting for a significant number of annual deaths. Oral cavity squamous cell carcinoma (OCSCC) and oropharyngeal squamous cell carcinoma (OPSCC) are the most common HNC subtypes. Current detection of those cancers involves painful incisional biopsies. There are no approved non-invasive options.

Etiologically, HNC is associated with tobacco and alcohol consumption, which can result in accumulation of cancer-related somatic mutations. Another factor known to contribute to OPSCC incidence is long-term HPV infection. Parodoxically, however, HPV positivity is associated with improved survival. Given that association, early detection and a determination of HPV status is critical for patient risk stratification, treatment management, and improved clinical outcomes.

Unfortunately, current OCSCC diagnostics are limited by the fact that early-stage oral cancer is often misdiagnosed. This leads to treatment delays and disease progression over time. Current intraoral and extraoral conventional visual and tactile exams may help reveal early signs of oral cancer. However, accurate determination is often elusive with those techniques, and biopsies must be performed to confirm disease presence.

Similarly, while HPV testing in patients with OPSCC is the standard of care in many institutions, there are still no HPV-specific diagnostic assays approved for OPSCC. In addition, many commercially available tests have recognized limitations. For example, testing for p16 overexpression is not always associated with the presence of hrHPV and does not provide quantitative assessment of HPV viral load. Detection of HPV-DNA by qPCR relies on external calibrators for absolute quantification, affecting reproducibility. While assays based on detection of E6/E7 mRNA (encoding viral oncoproteins) by RT-PCR depend on the quality and integrity of mRNA input. Importantly, assays designed to detect HPV in primary tumors (including ISH) do not allow serial monitoring of viral load over time.

There is, therefore, a need for non-invasive methods of detecting HNCs (e.g., OCSCC and OPSCC), preferably through testing of oral samples.

### SUMMARY

The present invention provides non-invasive diagnostic tests for accurate early-stage identification of disease. In a preferred embodiment, methods of the invention comprise liquid biopsy analysis of saliva for detection of biomarkers specific for HNC. In a preferred aspect, the invention comprises detecting HPV in a liquid biopsy sample. In further embodiments, the invention comprises detecting mutations in nucleic acid derived from a saliva sample. A combination of detecting nucleic acid mutations and HPV status further increases sensitivity. Finally, methods of the invention contemplate non-nucleic acid biomarkers, such as proteins. Methods of the invention are useful for diagnosis, prognosis, staging, and to inform effective treatment decisions and improve patient outcomes.

In preferred embodiments, methods of the invention involve targeted sequencing of genomic elements know or suspected to be associated with OCSCC tumorigenesis. To increase the clinical utility of assays of the invention, some embodiments further include HPV detection by incorporation of probes targeting high-risk HPV strains (e.g., HPV16/18). However, according to the invention, the presence of HPV in a saliva sample alone is diagnostic for the risk of HNC (specifically OPSCC). In certain embodiments, methods of the invention comprises sequencing a plurality of genes known or suspected to be involved in tumorogenesis.

In some specific embodiments, the invention comprises sequencing at least 3 genes known or suspected to be associated with HNC. In a preferred aspect, the invention comprises sequencing NOTCH1, TP53, FAT1, PIK3CA, CDKN2A, HRAS, and/or CASP8 genes or fragments thereof. Other embodiments comprise obtaining a body fluid sample, sequencing at least one of NOTCH1, TP53, FAT1, PIK3CA, CDKN2A, HRAS, and CASP8 and optionally HPV, and diagnosing NHC based on detection of one or more mutations in the sequencing step. Finally, in some embodiments, the invention contemplates the detection of a cancer-associated HPV strain in a saliva sample.

Embodiments of the invention comprise methods for detecting mutations in one or more OCSCC biomarkers in an oral rinse sample comprising saliva DNA. In preferred aspects, methods of the invention comprise sequencing nucleic acids (DNA or RNA) obtained from saliva. Multi-functional (somatic drivers/HPV) detection assays of the invention have clinical utility to patients with HPV+ disease and facilitate personalized treatment management based on tumor biology in addition to clinical risk factors.

Methods of the invention are applicable to any oropharyngeal cancer and include detection of biomarkers indicative of such cancers, including nucleic acids and proteins. A particular insight of the invention is that detection of cancer-associated biomarkers in addition to the detection of HPV subtypes provides increased sensitivity and specificity of detection in liquid biopsy samples. Any body fluid sample is useful in methods of the invention. Saliva, including salivary rinses, are a particularly good sample for use in methods of the invention due to its proximity to potential lesions. Further aspects and advantages of the invention are apparent to the skilled artisan in view of the following detailed description thereof.

### BRIEF DESCRIPTION OF THE DRAWINGS

FIG. 1 shows variants detected in 15 FFPE samples from OSCC patients.

FIG. 2 shows variants types in 15 FFPE samples from OSCC patients.

FIG. 3 shows genome alignment in 15 FFPE samples from HPV driven OPSCC patients.

FIG. 4 shows variants detected in 32 premalignant (oral dysplasia) FFPE samples.

FIG. 5 shows variants detected in 22 saliva samples from patients with oral dysplastic lesions.

FIG. 6 shows variants detected in 20 FFPE samples from OCSCC patients.

### DETAILED DESCRIPTION

### I. General Considerations

The invention provides methods for the detection of oropharyngeal cancer. Methods of the invention are also useful for determining prognosis of disease, staging of disease, and therapeutic selection. In particular, methods of the invention are implemented by detecting mutations in cancer-associated genomic elements and/or proteins in liquid biopsy samples, in particular saliva samples. The diagnostic and prognostic power of methods of the invention is increased by detection of HPV in the same sample in which mutations are detected. In certain embodiments, the presence of HPV in the sample alone is indicative of an increased risk of HNC; whereas absence of HPV in the sample is indicative of a low risk of HNC. Accordingly, an embodiment of the invention comprises detecting HPV in a saliva sample and assessing risk of HNC as the presence or absence of HPV in the sample. Where HPV is detected in the sample, identifying cancer-associated biomarkers provides additional diagnostic sensitivity and allows typing and stratification of cancer if present. Accordingly, in one embodiment, methods of the invention comprising obtaining a saliva sample and determining presence or absence of HPV in the sample. Further embodiments comprise detecting mutations in one or more cancer-associated biomarkers in the sample, including but not limited to NOTCH1, TP53, FAT1, PIK3CA, CDKN2A, HRAS, and CASP8. In yet other embodiments, protein biomarkers indicative of cancer are detected in the sample. In some aspects, detecting nucleic acid biomarkers in the sample comprises sequencing genomic DNA present in the sample. In other aspects, detection is accomplished by probe hybridization. In some aspects, sequences of suspected cancer biomarkers are compared to a reference database of wild-type sequences and/or mutations known to be associated with cancer. By comparison to wild-type sequences, novel sequence variants are identified and can be associated with the propensity for disease.

Methods of the invention are useful for the prognosis of disease and for therapeutic selection. For example, methods of the invention are useful to determine disease prognosis, severity, and/or staging based on sequences obtained in a saliva sample. In addition, HPV status is used to further inform disease status. Machine learning algorithms are useful to identify sequences that are correlated with disease, including prognosis and staging. Finally, algorithms are contemplated that allow sequences of putative biomarkers to be correlated with appropriate therapeutic selection.

What follows are methods used for implementing the invention and experimental results derived from their use.

### II. Nucleic Acid Detection

The current disclosure contemplates detection of mutations in genetic biomarkers. In one aspect, methods include constructing a cDNA library from a subject's DNA, such as saliva DNA. The terms “oligonucleotide,: ” “polynucleotide,” and “nucleic acid are used interchangeable and include linear oligomers of natural or modified monomers or linkages, including deoxyribonucleosides, ribonucleosides, α-anomeric forms thereof, peptide nucleic acids (PNAs), and the like, capable of specifically binding to a target (e.g. complementary or partially complementary) polynucleotide by way of a regular pattern of monomer-to-monomer interactions, such as Watson-Crick type of base pairing, base stacking, Hoogsteen or reverse Hoogsteen types of base pairing, or the like.

The nucleic acids of the disclosure may comprise or further comprise a barcode region that can identify the subject, gene, or biological sample. The barcode region can be a polynucleotide of at least, at most, or exactly 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200 or more (or any range derivable therein) nucleotides in length. The barcode may comprise or further comprise one or more universal PCR regions, adaptors, linkers, or a combination thereof. The barcode may represent a unique molecular identifier that may be used to determine whether a subject has a certain genetic mutation and/or the variant allele frequency of the genetic mutations.

Methods of the disclosure may include determining the identity of the barcode by determining the nucleotide sequence of the index region in order to identify which receptor(s) has been activated in a population of cells. The unique portions of the barcodes may be continuous along the length of the barcode sequence or the barcode may include stretches of nucleic acid sequence that is not unique to any one barcode. In one application, the unique portions of the barcodes may be separated by a stretch of nucleic acids that is removed by the cellular machinery during transcription into mRNA (e.g., an intron). The barcodes and/or index regions are quantified or determined by methods known in the art, including quantitative sequencing (e.g., using an Illumina® sequencer) or quantitative hybridization techniques (e.g., microarray hybridization technology or using a Luminex® bead system). Sequencing methods are further described herein.

### III. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, in some embodiments, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple cancer samples may be obtained for diagnosis by the methods described herein. In other cases, multiple samples, such as one or more samples from one tissue type (for example saliva) and one or more samples from another specimen (for example serum) may be obtained for diagnosis by the methods. In some cases, multiple samples such as one or more samples from one tissue type (e.g. saliva) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments, the biological sample is obtained by a physician, nurse, or other medical professional such as a medical technician, endocrinologist, cytologist, phlebotomist, radiologist, or a pulmonologist. The medical professional may indicate the appropriate test or assay to perform on the sample. In further aspects of the invention, the patient or subject may obtain a biological sample for testing without the assistance of a medical professional, such as obtaining a whole blood sample, a urine sample, a fecal sample, a buccal sample, or a saliva sample. Once obtained, samples may then be sent to a laboratory for processing.

In other cases, the sample is obtained by an invasive procedure including but not limited to biopsy, needle aspiration, endoscopy, or phlebotomy. Methods of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In some cases, the fine needle aspirate sampling procedure may be guided by the use of an ultrasound, X-ray, or other imaging device.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 M; or about 1 μM to 40 M; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 u M to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### V. Oral Cavity Squamous Cell Carcinoma (OCSCC)

The OCSCC may be further classified as a verrucous carcinoma, minor salivary gland carcinoma, or lymphoma. OCSCC may also exclude a verrucous carcinoma, minor salivary gland carcinoma, or lymphoma. The OCSCC may include or comprise cancer of the lip, tongue, palate, check, jaw, gum, soft palate, hard palate, uvula, or floor of the mouth. The OCSCC may exclude cancer of the lip, palate, check, jaw, gum, soft palate, hard palate, uvula, or floor of the mouth.

The OCSCC may be one that is linked to human papillomavirus (HPV) or may be independent of HPV, meaning that the subject has tested negative for a current or past HPV infection in the oral cavity.

In some aspects, the methods of treatment and detection may be for premalignant lesions of the oral cavity. A premalignant (or precancerous) lesion may be defined as “a morphologically altered tissue that has a greater than normal risk of malignant transformation.” There are several different types of premalignant lesion that occur in the mouth. Some oral cancers may begin as white patches (leukoplakia), red patches (erythroplakia) or mixed red and white patches (erythroleukoplakia or “speckled leukoplakia”). Other common premalignant lesions include oral submucous fibrosis and actinic cheilitis.

Methods of the disclosure may be performed in combination with one or more additional diagnostic procedures. Diagnostic procedures can include a CT scan, MRI, PET scan, endoscopy of the nasal cavity/pharynx, larynx, bronchus, and esophagus, biopsy, fine needle aspiration, CVTE, adjunctive screening, light-based screening (autofluoresence/tissue reflectance), and/or cytology screening.

The OCSCC may be one that is limited to a specific cancer stage, according to TNM classification. TNM classification for oral cancer is exemplified in the tables below:

T: Primary tumor

| TX | Primary tumor cannot be assessed | |
|---|---|---|
| Tis | Carcinoma in situ | |
| T1 | Tumor ≤2 cm with depth of invasion (DOI*) ≤5 mm | |
| T2 | Tumor ≤2 cm with DOI* >5 mm or tumor >2 cm and ≤4 cm with DOI* ≥10 mm | |
| T3 | Tumor >2 cm and ≤4 cm with DOI* >10 mm or tumor >4 cm with DOI* ≤10 mm | |
| T4 | Moderately advanced or very advanced local disease | |
| | T4a | Moderately advanced local disease, tumor >4 cm with DOI* >10 mm or |
| | | tumor invades adjacent structures only (cortical bone of the mandible or |
| | | maxilla (excluding superficial erosion of tooth socket alone in gingival |
| | | tumors) or involves the maxillary sinus or skin of the face) |
| | T4b | Very advanced local disease. Tumor invades masticator space, pterygoid |
| | | plates, or skull base and/or encases the internal carotid artery |

*DOI is depth of invasion and not tumor thickness.
N: Clinical Lymph nodes (separate classification for pathologic classification)

| NX | Regional lymph nodes cannot be assessed | |
|---|---|---|
| N0 | No regional lymph node metastasis | |
| N1 | Metastasis in a single ipsilateral lymph node, <3 cm and ENE(−) | |
| N2 | Metastasis in a single ipsilateral lymph node, ≤3 cm or smaller and ENE(+) or >3 | |
| | cm and ≤6 cm and ENE(−); or metastases in multiple ispsilateral lymph nodes, | |
| | none >6 cm and ENE(−); or in bilateral or contralateral lymph nodes(s), non >6 cm | |
| | ENE(−) | |
| | N2a | Metastasis in a single ipsilateral node <3 cm and ENE(+); or a single |
| | | ipsilatereral node ≥3 cm and <6 cm and ENE(−) |
| | N2b | Metastases in multiple ipsilateral nodes, <6 cm and ENE(−) |
| | N2c | Metastases in bilateral or contralateral lymph nodes(s); <6 cm and ENE(−) |
| N3 | Metastasis in a lymph node ≥6 cm and ENE(−); or metastasis in any nbodes(s) and | |
| | clinically overt ENE(+) | |
| | N3a | Metastasis in a lymph node ≥6 cm and ENE(−) |
| | N3b | Metastasis in any nodes(s) and clinically overt ENE(+) |

Note: A designation of “U” or “L” may be used for any N category to indicate metastasis
above (U) or below (L) the lower border of the cricoid. ENE(+/−) indicates presence or
absence of extranodal disease
M: Metastasis

| | |
|---|---|
| cM0 | No distant metastasis |
| cM1 | Distant metastasis |
| pM1 | Distant metastasis, microscopically confirmed |

TMN evaluation allows the person to be classified into a prognostic staging group:

AJCC Prognostic Stage Groups

| When T | And N | And M | Then the stage |
|---|---|---|---|
| is . . . | is . . . | is . . . | group is . . . |
| Tis | N0 | M0 | 0 |
| T1 | N0 | M0 | I |
| T2 | N0 | M0 | II |
| T3 | N0 | M0 | III |
| T1, T2, T3 | N1 | M0 | III |
| T4a | N0, N1 | M0 | IVA |
| T1, T2, T3, T4a | N2 | M0 | IVA |
| Any T | N3 | M0 | IVB |
| T4b | Any N | M0 | IVB |
| Any T | Any N | M1 | IVC |

The OCSCC in the methods of the disclosure may comprise T is, T1, T2, T3, T4a, T4b, N0, N1, N2, N3, M0, M1, stage 0, I, II, III, IVA, IVB, or IVC, or combinations thereof. In some aspects, the OCSCC excludes T is, T1, T2, T3, T4a, T4b, N0, N1, N2, N3, M0, M1, stage 0, I, II, III, IVA, IVB, or IVC.

In some aspects, the methods of the disclosure may be combined with a treatment for OCSCC. Treatments may include, for example, radiotherapy and chemotherapy, or surgery. The treatment may also include monoclonal antibody therapy, such as cetuximab

Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adrcocortical suppressants (e.g., taxol and mitotane). In some aspects, cisplatin is a particularly suitable chemotherapeutic agent.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

In some aspects, the may include radiotherapy, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### VI. Immunotherapy

In some aspects, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### A. Checkpoint Inhibitors and Combination Treatment

Aspects of the disclosure may include administration of immune checkpoint inhibitors, which are further described below.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### B. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### C. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

### D. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### E. Adoptive T-cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### VII. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

### B. DNA Sequencing

In some embodiments, DNA is analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### C. Detection Kits and Systems

One can recognize that based on the methods described herein, detection reagents, kits, and/or systems can be utilized to detect the genetic mutation related to the genetic signature for diagnosing an individual (the detection either individually or in combination). The reagents can be combined into at least one of the established formats for kits and/or systems as known in the art. As used herein, the terms “kits” and “systems” refer to embodiments such as combinations of at least one detection reagent, for example at least one selective oligonucleotide probe or at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In specific embodiments, the kit also comprises one or more therapeutic or prophylactic interventions in the event the individual is determined to be in need of.

### VIII. Probes

Sequencing probes useful for detecting mutations in NOTCH1, TP53, FAT1, PIK3CA, CDKN2A, HRAS, and/or CASP8 herein may include one or more probes provided in the Table below:

| | Probe | | |
|---|---|---|---|
| Gene/ | Pair | | |
| Chr | Name | F-sp | R-sp |
| NOT | NOTCH1 | GGTGTCCATGACCTTGTCAGTT | CTGTTGCACATGACTCCCTCC |
| CH1 | _46 | T (SEQ ID NO: 1) | (SEQ ID NO: 2) |
| NOT | NOTCH1 | GGAGGGCCCAGGAGAGTT (SEQ | CCAGCAGATGATCTTCCCCTAC |
| CH1 | _20 | ID NO: 3) | (SEQ ID NO: 4) |
| NOT | NOTCH1 | TGCAGTTTAGTAAGTGGGTAGC | GGACTGGACCTCAGGGAAGAG |
| CH1 | _54 | A (SEQ ID NO: 5) | (SEQ ID NO: 6) |
| NOT | NOTCH1 | GGTCAGGCCCTTGTGTCC (SEQ | CAACCTACCCCATCTGCTTCTTT |
| CH1 | _11 | ID NO: 7) | (SEQ ID NO: 8) |
| NOT | NOTCH1 | GAGGCCTGAGAGCTTCCT (SEQ | TGACCAACCGGCCTCCT (SEQ ID |
| CH1 | _37 | ID NO: 9) | NO: 10) |
| NOT | NOTCH1 | TAGGAGCCGACCTCGTTGT | GACCCTCTTGTCCCCTTGTCT |
| CH1 | _56 | (SEQ ID NO: 11) | (SEQ ID NO: 12) |
| FAT1 | FAT1_92 | TGAAGCAACTATCCTCATTTCC | GCAGGCAAATAAATTACACAAC |
| | | T (SEQ ID NO: 13) | C (SEQ ID NO: 14) |
| HRA | HRAS_1 | CCAGGAGACCGGCAGGG (SEQ | CTCCTGATCCCATCCCTCCTT |
| S | | ID NO: 15) | (SEQ ID NO: 16) |
| TP53 | TP53_13 | AATTTTCGCTTCCCACAGGTCT | GGGTTGGAAGTGTCTCATGCT |
| | | C (SEQ ID NO: 17) | (SEQ ID NO: 18) |
| TP53 | TP53_9 | CAGCTGCTCACCATCGCTATC | TATCTGTTCACTTGTGCCCTGAC |
| | | (SEQ ID NO: 19) | (SEQ ID NO: 20) |
| FAT1 | FAT1_59 | AGGTAGTAATGCACTTCCCCGT | GGCGTTTGATGTGGTTGTAGAA |
| | | T (SEQ ID NO: 21) | G (SEQ ID NO: 22) |
| FAT1 | FAT1_51 | CCAGAATCCCCATCCGTAGTTT | TCATTGACAGTGCAACAGGGAT |
| | | T (SEQ ID NO: 23) | T (SEQ ID NO: 24) |
| FAT1 | FAT1_10 | TCAAGTGCTTTCACTATCAATG | CAGGAGAACTCTGCAGCTAAGA |
| | 6 | TGT (SEQ ID NO: 25) | C (SEQ ID NO: 26) |
| FAT1 | FAT1_1 | GAAGCACTGCTGCAAAGAACA | CCCATGTCTGTGTACGCCTC |
| | | G (SEQ ID NO: 27) | (SEQ ID NO: 28) |
| FAT1 | FAT1_21 | CAGACACCTTTTCATCGCAGAA | GTGTGAGGAGGAACGACATAC |
| | | C (SEQ ID NO: 29) | AG (SEQ ID NO: 30) |
| FAT1 | FAT1_16 | CATAGCTTCTGGGTTTGCTGTTT | GAATGGAAACTATGCTCGCTTG |
| | | (SEQ ID NO: 31) | G (SEQ ID NO: 32) |
| FAT1 | FAT1_67 | GCTTTCTGAGTGATGATAGTTC | GAGTGAAATAACTTCTGTGCGT |
| | | C (SEQ ID NO: 33) | A (SEQ ID NO: 34) |
| FAT1 | FAT1_10 | CGGTCTTTGCAAGAAAGCCGTA | CAACTGCAGCCACGAGTACA |
| | | (SEQ ID NO: 35) | (SEQ ID NO: 36) |
| CDK | CDKN2 | TCAGGGTACAAATTCTCAGATC | GGGCTTCCTGGACACG (SEQ ID |
| N2A | A_3 | A (SEQ ID NO: 37) | NO: 38) |
| CDK | CDKN2 | TGTATCTGATAAAGAGCATACT | CAGGGGAATTGGAATCAGGTAG |
| N2A | A_5 | TCCA (SEQ ID NO: 39) | (SEQ ID NO: 40) |
| NOT | NOTCH1 | GAGCTCACGCCAAGGTG (SEQ | GCCCGCTGCACAGTAG (SEQ ID |
| CH1 | _4 | ID NO: 41) | NO: 42) |
| NOT | NOTCH1 | TGTTGCCCGCGTCCTC (SEQ ID | TTCACGGGCCCCGAATG (SEQ ID |
| CH1 | _25 | NO: 43) | NO: 44) |
| NOT | NOTCH1 | CAACCCTTACCCTAGGAGGGAC | TCACCTGTCTACCACCCCATC |
| CH1 | _31 | (SEQ ID NO: 45) | (SEQ ID NO: 46) |
| NOT | NOTCH1 | CTTCTTCCTCCGTGCCTTGAG | GCGCATGCATCACGACATC |
| CH1 | _9 | (SEQ ID NO: 47) | (SEQ ID NO: 48) |
| NOT | NOTCH1 | GCCACGGGGCTAGGGAA (SEQ | GGCTCTGCTCACTCCCTCT (SEQ |
| CH1 | _15 | ID NO: 49) | ID NO: 50) |
| NOT | NOTCH1 | CTGGGCCATCTCCAGAAGACAA | GGTGAGACTGACCTCTCTTCTC |
| CH1 | _59 | T (SEQ ID NO: 51) | C (SEQ ID NO: 52) |
| NOT | NOTCH1 | AGACATCCTGACCTCCCATCC | CGATTTGGGAGATCCCTCTGG |
| CH1 | _38 | (SEQ ID NO: 53) | (SEQ ID NO: 54) |
| NOT | NOTCH1 | TAGAAGGGGCTCTCGGATGTG | ACCGTCCTGTCTTCCCTCTC |
| CH1 | _26 | (SEQ ID NO: 55) | (SEQ ID NO: 56) |
| HRA | HRAS_6 | CAGCCCTATCCTGGCTGTG (SEQ | CAGGAGACCCTGTAGGAGGAC |
| S | | ID NO: 57) | (SEQ ID NO: 58) |
| NOT | NOTCH1 | GGGATGGCACCCCCTG (SEQ ID | CAGCCCCTCTCTGATTGTCC |
| CH1 | _19 | NO: 59) | (SEQ ID NO: 60) |
| NOT | NOTCH1 | GACCTTGATGGGCTGGGAC | CACCCAGCTGACCCCAATC (SEQ |
| CH1 | _32 | (SEQ ID NO: 61) | ID NO: 62) |
| TP53 | TP53_12 | AGATGGGTGAAAAGAGCAGTC | GAGACCTGTGGGAAGCGAAAA |
| | | AG (SEQ ID NO: 63) | T (SEQ ID NO: 64) |
| FAT1 | FAT1_24 | CGTCGTGAACTTCCCATCTGTT | TTTGCCCCTGGAGATTTTCATCA |
| | | A (SEQ ID NO: 65) | (SEQ ID NO: 66) |
| CDK | CDKN2 | CAAACAAAACAAGTGCCGAAT | CAGGTTCTTGGTGACCCTCC |
| N2A | A_9 | GC (SEQ ID NO: 67) | (SEQ ID NO: 68) |
| CDK | CDKN2 | ACAAACCCCTTCTGAAAACTCC | CCCGGGGTCGGGTAGA (SEQ ID |
| N2A | A_6 | C (SEQ ID NO: 69) | NO: 70) |
| PIK3 | PIK3CA_ | GTATTGCTTGGTAAAAGATTGG | AGGAAGTTGTATGGATCTAGAA |
| CA | 22 | C (SEQ ID NO: 71) | TAA (SEQ ID NO: 72) |
| TP53 | TP53_8 | TCTGTCATCCAAATACTCCACA | GGCCATCTACAAGCAGTCACA |
| | | CG (SEQ ID NO: 73) | (SEQ ID NO: 74) |
| FAT1 | FAT1_8 | TACTGTCATAGTCAAAGTCCCA | CTATTTCCTACACCCCGAGTATT |
| | | G (SEQ ID NO: 75) | (SEQ ID NO: 76) |
| FAT1 | FAT1_66 | TAAAAACTGGCGCGTTGTCATT | GGAGTTTCGTTGGGATGGTTAC |
| | | (SEQ ID NO: 77) | A (SEQ ID NO: 78) |
| FAT1 | FAT1_58 | AACGATAACTTCCGCTGAAAAG | TGTTAAAGTGGACACTGAGGTG |
| | | G (SEQ ID NO: 79) | G (SEQ ID NO: 80) |
| FAT1 | FAT1_93 | TTTACCTGAATACCAGTCGGAA | ATCACAGCTACAGATGGAGAAA |
| | | G (SEQ ID NO: 81) | A (SEQ ID NO: 82) |
| FAT1 | FAT1_50 | ACTTTTCCTCCAGCATCCTTAG | TGAAGTGGAACTAGCTGAAAAC |
| | | C (SEQ ID NO: 83) | G (SEQ ID NO: 84) |
| FAT1 | FAT1_33 | GTGGCACCATATTCACGGTACT | CACTGATTGACTCTGCTGATGG |
| | | (SEQ ID NO: 85) | G (SEQ ID NO: 86) |
| FAT1 | FAT1_2 | TCATAGTCACTCATCATGACCT | TGCCCAATTTTTATCCCCTCGAT |
| | | CG (SEQ ID NO: 87) | (SEQ ID NO: 88) |
| FAT1 | FAT1_10 | GCTTTGAACTGTGGAGAAGT | GCCAATGGTGACATAGCATCTT |
| | 0 | (SEQ ID NO: 89) | (SEQ ID NO: 90) |
| FAT1 | FAT1_45 | TGCATCTTTCACTTGGATACTA | CTCATCCGAGCAGAACATAGTG |
| | | ACA (SEQ ID NO: 91) | G (SEQ ID NO: 92) |
| CAS | CASP8_1 | CATCTATGGCACTGATGGACAG | CCATCCCCAGCAGAAAGTCAG |
| P8 | 3 | G (SEQ ID NO: 93) | (SEQ ID NO: 94) |
| NOT | NOTCH1 | GTGGGACCAGCGAGGAT (SEQ | CAAACATCCAGCAGCAGCAAA |
| CH1 | _3 | ID NO: 95) | (SEQ ID NO: 96) |
| NOT | NOTCH1 | CAGAGCTCCCAGGGTTTAGGA | AGGGAGCATGTGTAACATCAAC |
| CH1 | _41 | (SEQ ID NO: 97) | A (SEQ ID NO: 98) |
| NOT | NOTCH1 | AAGCAACCCACAGATGTTCCC | GGGCAAGTGCATCAACACG |
| CH1 | _48 | (SEQ ID NO: 99) | (SEQ ID NO: 100) |
| NOT | NOTCH1 | CAGGTGCCCCCGTTCTG (SEQ ID | CAGTGCCTGCCCTTCGAG (SEQ |
| CH1 | _55 | NO: 101) | ID NO: 102) |
| NOT | NOTCH1 | TCCCGCCCCACGACAG (SEQ ID | GGCATCGGTGTACGTCTGC (SEQ |
| CH1 | _12 | NO: 103) | ID NO: 104) |
| PIK3 | PIK3CA_ | AGATATGATGCAGCCATTGACC | AGTGTCCAAAATCTATATGAAA |
| CA | 35 | T (SEQ ID NO: 105) | CAGC (SEQ ID NO: 106) |
| PIK3 | PIK3CA_ | ACTTGATGCCCCCAAGAATCCT | GTTGAAAAAGCCGAAGGTCACA |
| CA | 2 | A (SEQ ID NO: 107) | A (SEQ ID NO: 108) |
| TP53 | TP53_4 | CAAATGCCCCAATTGCAGGTAA | TGCAGTTATGCCTCAGATTCAC |
| | | A (SEQ ID NO: 109) | T (SEQ ID NO: 110) |
| PIK3 | PIK3CA_ | TCTTTGTAACACTTCAAAAAGC | CATGATGTCTGGGTTCTCCC |
| CA | 29 | TA (SEQ ID NO: 111) | (SEQ ID NO: 112) |
| HRA | HRAS_3 | GGGTCCCTGGCTAGCTGT (SEQ | GTAGCCAGCTCTCGCTTTCC |
| S | | ID NO: 113) | (SEQ ID NO: 114) |
| FAT1 | FAT1_6 | CAGTAACACAACAAGCAAGCA | ACCGTCTTGGCCCATATACTTA |
| | | AAG (SEQ ID NO: 115) | C (SEQ ID NO: 116) |
| FAT1 | FAT1_18 | GGTTTGAAAGTCTTCCACTAAG | TCTGGTTTATTTTTTCCTCTGTG |
| | | ATGC (SEQ ID NO: 117) | TGA (SEQ ID NO: 118) |
| FAT1 | FAT1_81 | TTCTTTCTCGGTCTGGCTTTTCC | AGCATGAGGAAGAAGATGCTC |
| | | (SEQ ID NO: 119) | AC (SEQ ID NO: 120) |
| PIK3 | PIK3CA_ | TTTAATTTTGCACGATTCTTTTA | CATCCTTCTTCTCCTGTTTGAGA |
| CA | 25 | GAT (SEQ ID NO: 121) | (SEQ ID NO: 122) |
| PIK3 | PIK3CA_ | TTCCTTTTGGGGAAGAAAAGTG | TTGGATTTGATCCAGTAACACC |
| CA | 14 | T (SEQ ID NO: 123) | A (SEQ ID NO: 124) |
| PIK3 | PIK3CA_ | TTAAATGGAAACTTGCACCCTG | ACAGAGAAAACCATTACTTGTC |
| CA | 34 | T (SEQ ID NO: 125) | CA (SEQ ID NO: 126) |
| NOT | NOTCH1 | TCCCATCCCCCATCTAACTG | CTGTAACGAGGGCTCCAAC |
| CH1 | _50 | (SEQ ID NO: 127) | (SEQ ID NO: 128) |
| CAS | CASP8_7 | GGGAATCGGCAAAACCTACTCT | AAGATGTGCATGTGGTAGAAGG |
| P8 | | AA (SEQ ID NO: 129) | C (SEQ ID NO: 130) |
| CAS | CASP8_9 | TGCAATGGAAAGCAAGTCCTCT | TGTTCCATTCCTGTCCCTAATGC |
| P8 | | (SEQ ID NO: 131) | (SEQ ID NO: 132) |
| FAT1 | FAT1_32 | TTGGTGGAATGAAGGGTTTCCT | TGACATAAATGACAACCCCCCT |
| | | C (SEQ ID NO: 133) | G (SEQ ID NO: 134) |
| TP53 | TP53_1 | AAAGACCCAAAACCCAAAATG | CAAAGCATTGGTCAGGGAAAA |
| | | GC (SEQ ID NO: 135) | GG (SEQ ID NO: 136) |
| FAT1 | FAT1_64 | ACTGTTACATTCGCTGCATACT | GAATGTCCCACTGGTGATTCGA |
| | | CA (SEQ ID NO: 137) | G (SEQ ID NO: 138) |
| FAT1 | FAT1_73 | GAAGTATAGAGAGAGCCGGTT | TGTCATCCATTACAGGTATTCAT |
| | | G (SEQ ID NO: 139) | CA (SEQ ID NO: 140) |
| FAT1 | FAT1_57 | CGTCTGTGATGCTGTAGAACAC | AAGCAATTTGAGCTTGACACCT |
| | | T (SEQ ID NO: 141) | T (SEQ ID NO: 142) |
| FAT1 | FAT1_10 | CACTGCTCCCATACAACTTCAT | TCATACAGCGTTTCTTTACCTGA |
| | 3 | (SEQ ID NO: 143) | (SEQ ID NO: 144) |
| FAT1 | FAT1_38 | ACTCCATCATTACCTGTGTCTG | CTTGATGTTTGTGCTGCCTGTTA |
| | | G (SEQ ID NO: 145) | (SEQ ID NO: 146) |
| FAT1 | FAT1_4 | GAACCTCTGCCGGTTTCTTGAT | ACTATGAGGTGATTGATGAGCA |
| | | G (SEQ ID NO: 147) | GAC (SEQ ID NO: 148) |
| FAT1 | FAT1_49 | CATAGGTGATGTCGGCATTGGA | TTGGAAAAACTTGATCGAGAAA |
| | | (SEQ ID NO: 149) | CC (SEQ ID NO: 150) |
| FAT1 | FAT1_89 | ATACTTTCACAACGACAGTGGA | GAAGACAAGGAGGTACATAGT |
| | | (SEQ ID NO: 151) | GA (SEQ ID NO: 152) |
| FAT1 | FAT1_98 | GCACGTTCTCATCAAAAGCAG | GCTTATTCCCATTTGAGGTATGT |
| | | (SEQ ID NO: 153) | T (SEQ ID NO: 154) |
| FAT1 | FAT1_22 | TCCAATGATTTCCTCAATGTCA | ATCCGCTTTGCCAACCT (SEQ ID |
| | | G (SEQ ID NO: 155) | NO: 156) |
| CAS | CASP8_1 | TGATATTCTCACCATCCTGACT | ACAAAACAAACAAAATAGCAC |
| P8 | 6 | G (SEQ ID NO: 157) | CA (SEQ ID NO: 158) |
| CAS | CASP8_2 | TGGTACTTTTCTTCCTTATCTGA | ACAGATTGCTTTCCTCCAACATT |
| P8 | | ACA (SEQ ID NO: 159) | (SEQ ID NO: 160) |
| NOT | NOTCH1 | CCTAGGGGTAAGAGCAGGG | GCGTGGACCAGGTGGG (SEQ ID |
| CH1 | _28 | (SEQ ID NO: 161) | NO: 162) |
| NOT | NOTCH1 | GGTGCTGGTGCCAGAG (SEQ ID | GGAGTCACCCCATGGCTA (SEQ |
| CH1 | _7 | NO: 163) | ID NO: 164) |
| NOT | NOTCH1 | CAGTTGGGGCCAGTGTAGC | GGGGACCCACCAATGCC (SEQ |
| CH1 | _34 | (SEQ ID NO: 165) | ID NO: 166) |
| NOT | NOTCH1 | GCGTTGTCGCGGTCCT (SEQ ID | GCCAGTCCTAAGTCTTCCTGTG |
| CH1 | _45 | NO: 167) | (SEQ ID NO: 168) |
| NOT | NOTCH1 | GAGCAGCGAGGCCTTCA (SEQ | GGCACGCTGGTGGTGG (SEQ ID |
| CH1 | _21 | ID NO: 169) | NO: 170) |
| PIK3 | PIK3CA_ | TCTTCCAAATCTACAGAGTTCC | ACTATGAGGTGAATTGAGGTCC |
| CA | 4 | CT (SEQ ID NO: 171) | C (SEQ ID NO: 172) |
| PIK3 | PIK3CA_ | TGATGTTGATGGCTAAAGAAAG | AAAATAATAAGCATCAGCATTT |
| CA | 10 | C (SEQ ID NO: 173) | GAC (SEQ ID NO: 174) |
| PIK3 | PIK3CA_ | TTTTCTCAATGATGCTTGGCTCT | TGTGGAATCCAGAGTGAGCTTT |
| CA | 39 | (SEQ ID NO: 175) | C (SEQ ID NO: 176) |
| PIK3 | PIK3CA_ | TGCTTTTTCTGTAAATCATCTGT | TCCATTTTAGCACTTACCTGTGA |
| CA | 18 | GAA (SEQ ID NO: 177) | CT (SEQ ID NO: 178) |
| FAT1 | FAT1_19 | AAATGGTAAGAAACAATGAAA | TTTTCCCTTTCTCCCCTCTTTGA |
| | | CCGA (SEQ ID NO: 179) | (SEQ ID NO: 180) |
| NOT | NOTCH1 | GTCAGGCGTGTTGTTCTCACAG | CCCCTCACACTCACCCTTC (SEQ |
| CH1 | _36 | (SEQ ID NO: 181) | ID NO: 182) |
| PIK3 | PIK3CA_ | AGTTTTTAGTTGAGCAAATGAG | AGGAGAATGAGAGAGAGAAGC |
| CA | 28 | GC (SEQ ID NO: 183) | AT (SEQ ID NO: 184) |
| TP53 | TP53_2 | GTCAGCTGCCTTTGACCATGAA | TACTTACTTCTCCCCCTCCTCTG |
| | | G (SEQ ID NO: 185) | (SEQ ID NO: 186) |
| TP53 | TP53_5 | GTGAATCTGAGGCATAACTGCA | CCTTACTGCCTCTTGCTTCTCTT |
| | | C (SEQ ID NO: 187) | (SEQ ID NO: 188) |
| PIK3 | PIK3CA_ | GGAAAATGACAAAGAACAGCT | CTGAGATCAGCCAAATTCAGTT |
| CA | 19 | CA (SEQ ID NO: 189) | A (SEQ ID NO: 190) |
| NOT | NOTCH1 | TTGCACTCATTGACCTCAGACA | TCAACAGACAGGGAAATCGAG |
| CH1 | _42 | G (SEQ ID NO: 191) | GT (SEQ ID NO: 192) |
| CAS | CASP8_6 | AAAACCAGTAGGGCTCAATCC | TAGAGTAGGTTTTGCCGATTCC |
| P8 | | AG (SEQ ID NO: 193) | C (SEQ ID NO: 194) |
| FAT1 | FAT1_71 | TCGAGTACAGCACTTCAGCATT | GCACCAATGGGATTGATAATGT |
| | | T (SEQ ID NO: 195) | GTC (SEQ ID NO: 196) |
| FAT1 | FAT1_31 | CTGATGACTGTCGTGTAGGTGT | ATAGTGGGAAAATGTGGTGGCA |
| | | C (SEQ ID NO: 197) | T (SEQ ID NO: 198) |
| FAT1 | FAT1_75 | TCTCCATCCTCCTCTGACAAGT | ACAGAGTGTTATCCTGTGACCT |
| | | G (SEQ ID NO: 199) | G (SEQ ID NO: 200) |
| FAT1 | FAT1_47 | AACAGTCCCACTATGTTCTGCT | CTGTCCGGCGTAATCACTACAA |
| | | C (SEQ ID NO: 201) | A (SEQ ID NO: 202) |
| FAT1 | FAT1_37 | TCAGAATACATTTGAAAAGGGC | AGACGTCCTTCCTGGAAAATTG |
| | | AAA (SEQ ID NO: 203) | A (SEQ ID NO: 204) |
| FAT1 | FAT1_55 | CCAAACATCTGGTATGAGATTC | CTGGACTTTGAGGCCCACC (SEQ |
| | | CTCT (SEQ ID NO: 205) | ID NO: 206) |
| FAT1 | FAT1_97 | ATCAGTTCGTAGTCCAGGTTTT | AAGGTCTTGGTGAAAGTCTTAG |
| | | C (SEQ ID NO: 207) | G (SEQ ID NO: 208) |
| FAT1 | FAT1_63 | TTGCCTTCGGTGATGGAGTAAA | CACTTTACCGTCCAAGTGCATG |
| | | T (SEQ ID NO: 209) | A (SEQ ID NO: 210) |
| FAT1 | FAT1_87 | AGCAAGTAGAAGACAGAGAAA | GAAAGTCCGAGAGGATCTTCCA |
| | | CT (SEQ ID NO: 211) | (SEQ ID NO: 212) |
| FAT1 | FAT1_10 | CTGGTCCTTATAGCTGTGTTTTC | AAGCTGAAGAGTACATTCTCGG |
| | 5 | (SEQ ID NO: 213) | (SEQ ID NO: 214) |
| FAT1 | FAT1_3 | GCCTCGAAGTGTCTTTGATACC | CGCCCGAATTCAGCAATCAGTT |
| | | C (SEQ ID NO: 215) | (SEQ ID NO: 216) |
| FAT1 | FAT1_12 | TGCCAGTCAATATGACGCTATG | TCTGTTTTGCTTTCTGTTTTGCT |
| | | A (SEQ ID NO: 217) | G (SEQ ID NO: 218) |
| FAT1 | FAT1_80 | ATGCTGTAGGAGATTTCTGCAT | TTACTGTGACAGACAATGGTAG |
| | | T (SEQ ID NO: 219) | T (SEQ ID NO: 220) |
| FAT1 | FAT1_41 | TCATCCCTTAAATCCCCAATTT | CAACGATAGTCCACCACGATTC |
| | | CCA (SEQ ID NO: 221) | A (SEQ ID NO: 222) |
| CAS | CASP8_1 | AACTACAGAACTATCTCTGAGC | GCACCATCAATCAGAAGGGAA |
| P8 | 5 | AC (SEQ ID NO: 223) | GA (SEQ ID NO: 224) |
| NOT | NOTCH1 | GCACCTGCTGGGTCTG (SEQ ID | GTTTGAATGGTCAATGCGAGT |
| CH1 | _5 | NO: 225) | (SEQ ID NO: 226) |
| NOT | NOTCH1 | CAGGGATTGGACAGGCACTC | GTGTGGCGGGCTTGGG (SEQ ID |
| CH1 | _29 | (SEQ ID NO: 227) | NO: 228) |
| NOT | NOTCH1 | CACGGGCTCCTCGAACTACAT | CGCTCAGCCTCACTTCTCG (SEQ |
| CH1 | _16 | (SEQ ID NO: 229) | ID NO: 230) |
| NOT | NOTCH1 | TACCCAGCGAGCACTCAT (SEQ | GGGTGGTGTGCCATGC (SEQ ID |
| CH1 | _51 | ID NO: 231) | NO: 232) |
| PIK3 | PIK3CA_ | ATGAAAAACCTTACAGGAAAT | GCACTATTTATAACCCAAAGGG |
| CA | 9 | GG (SEQ ID NO: 233) | A (SEQ ID NO: 234) |
| PIK3 | PIK3CA_ | TGGTTTTGTTTCTAAATTACAG | CGACAATAGGACTCCAAAAGC |
| CA | 24 | GT (SEQ ID NO: 235) | (SEQ ID NO: 236) |
| PIK3 | PIK3CA_ | AGACGATGGACAAGTAATGGT | TTGTGCATTCTTGGGCTCCTTTA |
| CA | 36 | TT (SEQ ID NO: 237) | (SEQ ID NO: 238) |
| PIK3 | PIK3CA_ | CCCTATTGGTGTTACTGGATCA | GGGAATAGCTAAATCCTGCTTC |
| CA | 16 | AA (SEQ ID NO: 239) | T (SEQ ID NO: 240) |
| PIK3 | PIK3CA_ | CCAGTGTGTGAATTTGATATGG | ACACAGGTAGAAGACTGCACTA |
| CA | 5 | TT (SEQ ID NO: 241) | (SEQ ID NO: 242) |
| PIK3 | PIK3CA_ | CATCATTTGCTCCAAACTGACC | TGAAATACTCCAAAGCCTCTTG |
| CA | 38 | A (SEQ ID NO: 243) | CT (SEQ ID NO: 244) |
| CAS | CASP8_1 | ACCAAATGAAAAGCAAACCTC | ACATGGCCCTTTTGGTATAGCTT |
| P8 | 0 | GG (SEQ ID NO: 245) | (SEQ ID NO: 246) |
| CAS | CASP8_5 | GCCCACTGTGACTCCATATATC | GGCTAACCAAGTCTCAGGACTA |
| P8 | | A (SEQ ID NO: 247) | A (SEQ ID NO: 248) |
| TP53 | TP53_11 | GTAGGTTTTCTGGGAAGGGACA | TCTGACTGCTCTTTTCACCCATC |
| | | G (SEQ ID NO: 249) | (SEQ ID NO: 250) |
| PIK3 | PIK3CA_ | ACCATGACTGTGTACCAGAACA | AACTTGTTACTCACCTTATACTG |
| CA | 7 | A (SEQ ID NO: 251) | ACT (SEQ ID NO: 252) |
| CAS | CASP8_1 | CCAGCTGTGGTCTGTGAATTAC | CCATCAGTGCCATAGATGATGC |
| P8 | 1 | T (SEQ ID NO: 253) | C (SEQ ID NO: 254) |
| FAT1 | FAT1_77 | GATGTCAAACCAAAGGGGTAT | TGTTTTTAATCCCCTCTGCCCAA |
| | | GC (SEQ ID NO: 255) | (SEQ ID NO: 256) |
| PIK3 | PIK3CA_ | AGAAAGCAGCAGTCTAGGATG | GTTTCCTAGTTGATGAGCAGGG |
| CA | 27 | TA (SEQ ID NO: 257) | T (SEQ ID NO: 258) |
| NOT | NOTCH1 | CTCCCGCCGCTTCTTCTTG (SEQ | TGATGTCCGGGCACCTG (SEQ ID |
| CH1 | _18 | ID NO: 259) | NO: 260) |
| TP53 | TP53_3 | AAGCAGGCTAGGCTAAGCTAT | AGCTAACTAACTTCAGAACACC |
| | | GA (SEQ ID NO: 261) | AACT (SEQ ID NO: 262) |
| FAT1 | FAT1_69 | ATCATACTCCGCTTGGTTACTT | TATTGACATCCCCTGAGTTTCTG |
| | | CG (SEQ ID NO: 263) | C (SEQ ID NO: 264) |
| FAT1 | FAT1_99 | ATAGAAATGAGACCAGTGTTGT | CCCCAGTTCTCTTCTGTTAAAGT |
| | | A (SEQ ID NO: 265) | (SEQ ID NO: 266) |
| FAT1 | FAT1_72 | CACAAAGGCCTCAGGACCAAA | AGATACAGCGCCAGAAACAGA |
| | | TG (SEQ ID NO: 267) | AA (SEQ ID NO: 268) |
| FAT1 | FAT1_26 | TTCAAATAGAAGAGGGGAACC | GGATTCTTCCCATAACGGTCCA |
| | | AC (SEQ ID NO: 269) | C (SEQ ID NO: 270) |
| FAT1 | FAT1_44 | ATCAGATGCCCTGATCTGAATT | AGACTGAAGTTGGAGAAGAGT |
| | | A (SEQ ID NO: 271) | (SEQ ID NO: 272) |
| FAT1 | FAT1_39 | GAATTACACCACAGCGCCAAAT | TGTGAAGAAACCTCTAGACAGG |
| | | G (SEQ ID NO: 273) | GA (SEQ ID NO: 274) |
| FAT1 | FAT1_13 | ACAGCAAAACAGAAAGCAAAA | CCCACTGTGAGATAAGCGTCAA |
| | | CA (SEQ ID NO: 275) | T (SEQ ID NO: 276) |
| FAT1 | FAT1_82 | GCTAAGAATACTGGTTAATGGG | acctggccAAGAGTGTGATTTTA |
| | | AACA (SEQ ID NO: 277) | (SEQ ID NO: 278) |
| FAT1 | FAT1_60 | CTACAATGACCTTCACGACAAC | AGGCCGAAACATTAGCTGTCAT |
| | | G (SEQ ID NO: 279) | T (SEQ ID NO: 280) |
| FAT1 | FAT1_34 | AGCTTTCAAAGAGAGGGTGTAT | AATCTTTTGACATGTTTTTATGT |
| | | (SEQ ID NO: 281) | CTT (SEQ ID NO: 282) |
| FAT1 | FAT1_53 | ACTGCAACTTGTCTATGTCTGA | GAACCCTGGATTACGAGCAGTC |
| | | AC (SEQ ID NO: 283) | (SEQ ID NO: 284) |
| FAT1 | FAT1_91 | ATTATCATTGGCATCGACAACC | CATGAACTCCACTGACCTTGA |
| | | A (SEQ ID NO: 285) | (SEQ ID NO: 286) |
| NOT | NOTCH1 | GGACGGAGACTGCTGGA (SEQ | TGCAGGGCAAGAAGGTC (SEQ |
| CH1 | _8 | ID NO: 287) | ID NO: 288) |
| NOT | NOTCH1 | GATGCACTGGAACTCCCCAAT | TTGCAGGGGCCAGGGT (SEQ ID |
| CH1 | _49 | (SEQ ID NO: 289) | NO: 290) |
| NOT | NOTCH1 | GGCTGCTGGCACAGTCAT (SEQ | CACCACTGCGAGACCAACA |
| CH1 | _44 | ID NO: 291) | (SEQ ID NO: 292) |
| NOT | NOTCH1 | AGTGGCCGTCACTGAAGTA | TCTTGTGCCACATCCTGGACTA |
| CH1 | _24 | (SEQ ID NO: 293) | (SEQ ID NO: 294) |
| PIK3 | PIK3CA_ | TCTTCGTGATTTGTAGGAGTCA | TCAGCGGTATAATCAGGAGTTT |
| CA | 13 | TT (SEQ ID NO: 295) | T (SEQ ID NO: 296) |
| PIK3 | PIK3CA_ | CACTCAAAGAGTACCTTGTTCC | TGTAGAAATGGGGTCTTGCTT |
| CA | 12 | A (SEQ ID NO: 297) | (SEQ ID NO: 298) |
| PIK3 | PIK3CA_ | CCCCCTCCATCAACTTCTTC | TTAGAAAGGGACAACAGTTAAG |
| CA | 3 | (SEQ ID NO: 299) | C (SEQ ID NO: 300) |
| PIK3 | PIK3CA_ | CAGAAACTGACCCTGATTTGTT | CCAGCCAAACATAAACAAAAGT |
| CA | 23 | (SEQ ID NO: 301) | AT (SEQ ID NO: 302) |
| PIK3 | PIK3CA_ | GTGACTGTGTGGGACTTATTGA | AACTCAGAGGAATACACAAAC |
| CA | 33 | GG (SEQ ID NO: 303) | ACC (SEQ ID NO: 304) |
| FAT1 | FAT1_70 | AGAAACAGACTTTTAACAACTG | CAAGGATTGTGGTCAATGTCAG |
| | | CAA (SEQ ID NO: 305) | C (SEQ ID NO: 306) |
| CDK | CDKN2 | ATGAAAACTACGAAAGCGGGG | AGTATTTCAATGCCGGTAGGGA |
| N2A | A_1 | TG (SEQ ID NO: 307) | C (SEQ ID NO: 308) |
| CDK | CDKN2 | CAGCTCCTCAGCCAGGTC (SEQ | GGCTCTGACCATTCTGTTCTCTC |
| N2A | A_4 | ID NO: 309) | (SEQ ID NO: 310) |
| CDK | CDKN2 | CCTACCTGGTCTTCTAGGAAGC | GAGCTCGGCCCTGGAG (SEQ ID |
| N2A | A_10 | G (SEQ ID NO: 311) | NO: 312) |
| FAT1 | FAT1_35 | GCTGAATCTGGGGTGTGTTTTG | GATTCTGCCAAGCCAGTATTGT |
| | | (SEQ ID NO: 313) | G (SEQ ID NO: 314) |
| NOT | NOTCH1 | ACGCCTGCATGGTGTGC (SEQ ID | TTTCTGTGAGGAGGACATCAAC |
| CH1 | _35 | NO: 315) | G (SEQ ID NO: 316) |
| NOT | NOTCH1 | CGGCAGGGGTTGGTGAG (SEQ | GAACTAACTGCCCTGGCACATC |
| CH1 | _58 | ID NO: 317) | (SEQ ID NO: 318) |
| NOT | NOTCH1 | CCGAAGGCTTGGGAAAGGAAG | CTGTGGACAACACCCCCAG |
| CH1 | _1 | (SEQ ID NO: 319) | (SEQ ID NO: 320) |
| HRA | HRAS_5 | TTGTTGATGGCAAACACACACA | CATGAGAGGTACCAGGGAGAG |
| S | | G (SEQ ID NO: 321) | G (SEQ ID NO: 322) |
| CDK | CDKN2 | GTGGCCAGCCAGTCAG (SEQ ID | CCAGAGGATTTGAGGGACAG |
| N2A | A_8 | NO: 323) | (SEQ ID NO: 324) |
| FAT1 | FAT1_7 | TGGACAGAATTTGTAACCTCAC | ATCCCGAATTCAGCACTTTTAA |
| | | T (SEQ ID NO: 325) | C (SEQ ID NO: 326) |
| FAT1 | FAT1_94 | GGCAGCCACTGTTATGTTGATA | AACCACTGTTTCTGCTATTGATG |
| | | (SEQ ID NO: 327) | (SEQ ID NO: 328) |
| TP53 | TP53_6 | CCCAGGGGTCAGAGGC (SEQ ID | CCTGCTTGCCACAGGTCT (SEQ |
| | | NO: 329) | ID NO: 330) |
| FAT1 | FAT1_15 | CCCTTAAATGTACACACGTGCA | ATGGTTTCAGGGGTTGTATGGA |
| | | G (SEQ ID NO: 331) | C (SEQ ID NO: 332) |
| FAT1 | FAT1_20 | AAAGGCCGTCTGAAACCATCTG | TGACTGACATTGAGGAAATCAT |
| | | (SEQ ID NO: 333) | TGGA (SEQ ID NO: 334) |
| FAT1 | FAT1_79 | CTGCTGAAAACCTCTTGGACGA | GAGAAAGAAATGCCAGACGGG |
| | | (SEQ ID NO: 335) | AG (SEQ ID NO: 336) |
| FAT1 | FAT1_88 | CTAAATCAGGATCGTGGGCTTC | TTAACATCGCACGCCCTCT (SEQ |
| | | (SEQ ID NO: 337) | ID NO: 338) |
| FAT1 | FAT1_46 | CTCTGTCTGTCAATCACAAAGG | AAAAGAATCTGTTGTTCTTGTCT |
| | | (SEQ ID NO: 339) | ATG (SEQ ID NO: 340) |
| FAT1 | FAT1_10 | CACGCTGGATGTGTAATGTAAA | ACGATTCTGATTTCTTCTGTTTC |
| | 7 | (SEQ ID NO: 341) | (SEQ ID NO: 342) |
| FAT1 | FAT1_28 | CATTTCAAAGAACCCCAGGACC | CTGTCGGCCTTGTGAAAATGAA |
| | | A (SEQ ID NO: 343) | C (SEQ ID NO: 344) |
| FAT1 | FAT1_52 | CTCCATCAGACACTGACAGGTT | TATGAAGCCAGAATTAGCGAGC |
| | | A (SEQ ID NO: 345) | A (SEQ ID NO: 346) |
| FAT1 | FAT1_10 | ATCCACTGTCACAATTGCATA | AGTGTTAACTGGTAGACTTGAT |
| | 2 | (SEQ ID NO: 347) | T (SEQ ID NO: 348) |
| FAT1 | FAT1_61 | TAAATCTGCGATCTGGGTTGAG | GTTAAGAAGCCGCTACGAGCTA |
| | | G (SEQ ID NO: 349) | A (SEQ ID NO: 350) |
| NOT | NOTCH1 | CTCCAGCACAGGCTCCG (SEQ | TGCTGGACATGCCGAGTG (SEQ |
| CH1 | _39 | ID NO: 351) | ID NO: 352) |
| NOT | NOTCH1 | CAGGAAGTGGAAGGAGCTGTT | GAGGCCCCCCGAGGAAG (SEQ |
| CH1 | _22 | G (SEQ ID NO: 353) | ID NO: 354) |
| NOT | NOTCH1 | GGCCAGGGTGCAGACG (SEQ ID | GTTCGTTTCTGTCCCAAGTCCAC |
| CH1 | _47 | NO: 355) | (SEQ ID NO: 356) |
| NOT | NOTCH1 | CCTGAGGAGGGCAGGT (SEQ ID | TCATCTCCGACTTCATCTACCA |
| CH1 | _13 | NO: 357) | (SEQ ID NO: 358) |
| NOT | NOTCH1 | CCATGGATGGCCAACACCAG | TCACAGTGGGGTGTGGGAG |
| CH1 | _30 | (SEQ ID NO: 359) | (SEQ ID NO: 360) |
| PIK3 | PIK3CA_ | GGATGTATTTGAAGCACCTGAA | GAGGCAGTGGAACTTGGT (SEQ |
| CA | 26 | T (SEQ ID NO: 361) | ID NO: 362) |
| PIK3 | PIK3CA_ | TGAAAGAGAGATGGTGATTGC | TCAGAGGATAGCAACATACTTC |
| CA | 6 | AT (SEQ ID NO: 363) | G (SEQ ID NO: 364) |
| PIK3 | PIK3CA_ | AAGGCAGTGTTTTAGATGGCTC | TATTTTTCCAAGCACCGAACAG |
| CA | 21 | A (SEQ ID NO: 365) | C (SEQ ID NO: 366) |
| CDK | CDKN2 | GTGAAGATGTGGCCTTTCCCTT | GCAGAAGCCAGAGCACATGAA |
| N2A | A_2 | C (SEQ ID NO: 367) | TA (SEQ ID NO: 368) |
| CDK | CDKN2 | GATTCCAATTCCCCTGCAAAC | GGGGAGCAGCATGGAG (SEQ ID |
| N2A | A_7 | (SEQ ID NO: 369) | NO: 370) |
| NOT | NOTCH1 | TTATCCTGGGTGCAGGAGGG | GCTACAACTGCGTGTGTGTCAA |
| CH1 | _52 | (SEQ ID NO: 371) | (SEQ ID NO: 372) |
| TP53 | TP53_10 | CCAGGCATTGAAGTCTCATGGA | GTCCAGATGAAGCTCCCAGAAT |
| | | A (SEQ ID NO: 373) | G (SEQ ID NO: 374) |
| CAS | CASP8_1 | ACTGCACAGTAGAGCAAATCTA | CCCTGACAAGCCTGAATAAAAA |
| P8 | 2 | T (SEQ ID NO: 375) | A (SEQ ID NO: 376) |
| FAT1 | FAT1_76 | TTGTGCCCAAAACTCATCAAAC | ATTGAATGGATCTCCAAGCCCA |
| | | C (SEQ ID NO: 377) | A (SEQ ID NO: 378) |
| FAT1 | FAT1_27 | CTCCTTCCTCTTGATGGCAGAT | AGATTTGGCTGTGTTCCAGGAT |
| | | G (SEQ ID NO: 379) | T (SEQ ID NO: 380) |
| NOT | NOTCH1 | CAAGTACCTCAAGTTGCCTGGG | CAGACTATGCCTGCAGCTGTG |
| CH1 | _57 | (SEQ ID NO: 381) | (SEQ ID NO: 382) |
| PIK3 | PIK3CA_ | AGTGTCGAATTATGTCCTCTGC | AGCTAAATTCATGCATCATAAG |
| CA | 30 | AA (SEQ ID NO: 383) | CTC (SEQ ID NO: 384) |
| PIK3 | PIK3CA_ | TAAACGAGAACGTGTGCCATTT | TGTTTCTAATAGAGCAGCCAGA |
| CA | 37 | G (SEQ ID NO: 385) | ACT (SEQ ID NO: 386) |
| CAS | CASP8_1 | AAGCCCTGCTGAATTTGCT (SEQ | TTTCCTGTTGAAGAGGCCACC |
| P8 | | ID NO: 387) | (SEQ ID NO: 388) |
| FAT1 | FAT1_11 | AAAATATCCCTGCAACAAACAC | CTCTCCTCATTCCTGACTTCCTT |
| | | A (SEQ ID NO: 389) | (SEQ ID NO: 390) |
| FAT1 | FAT1_29 | CAGACAAGAGCACAAGGCATT | CTCCTAAACAGGAGTGTGGTGT |
| | | T (SEQ ID NO: 391) | G (SEQ ID NO: 392) |
| FAT1 | FAT1_42 | CATCCTCACTCACAGTCCCTTT | CAGTCACAAAGTGTGGAAGTCA |
| | | A (SEQ ID NO: 393) | TT (SEQ ID NO: 394) |
| FAT1 | FAT1_5 | AAATCACTTTCGATGTCGTAGC | TTCCATGGCACAAAAGCTTAGG |
| | | C (SEQ ID NO: 395) | A (SEQ ID NO: 396) |
| FAT1 | FAT1_65 | ATAGCAAAATATGTGTGTACAG | ACATGGCTGGTTTGTCCACTAA |
| | | ATGG (SEQ ID NO: 397) | T (SEQ ID NO: 398) |
| FAT1 | FAT1_96 | TGTCCCTTCACAATTTATTTTCT | CGTGACATACAGTATCGCAAAT |
| | | CA (SEQ ID NO: 399) | (SEQ ID NO: 400) |
| FAT1 | FAT1_90 | TGAAAATGTGTCTGTGTCTGTA | ATATTACCGTCTATGACCTTGG |
| | | A (SEQ ID NO: 401) | G (SEQ ID NO: 402) |
| FAT1 | FAT1_10 | GAGCTTGGTCTCTAGGTAATCA | TGCTGGATACAAATGACTTGAG |
| | 4 | A (SEQ ID NO: 403) | A (SEQ ID NO: 404) |
| FAT1 | FAT1_56 | TGATGTCCACAAATACTTCAGC | CAGAGATTGCAGAGAGCATCCA |
| | | A (SEQ ID NO: 405) | G (SEQ ID NO: 406) |
| FAT1 | FAT1_48 | ACCCATTATCCACAGCTCTAAC | GAAGTGAATATCGGGTCCAGTG |
| | | A (SEQ ID NO: 407) | C (SEQ ID NO: 408) |
| FAT1 | FAT1_17 | GGCCACCAAAAAACACATAGT | TGTGAACAGTCATTGCTTTTATC |
| | | T (SEQ ID NO: 409) | C (SEQ ID NO: 410) |
| FAT1 | FAT1_23 | TCTGTATGTCGTTCCTCCTCACA | GGCAAGCTGATAGCACACAAA |
| | | (SEQ ID NO: 411) | AA (SEQ ID NO: 412) |
| FAT1 | FAT1_85 | AATGGAAAAGGGCATCAAATG | GAGGTGGTTGATGTGAATGAGA |
| | | AG (SEQ ID NO: 413) | A (SEQ ID NO: 414) |
| FAT1 | FAT1_84 | TGGACCACAGATACATCTTTAG | GATGTGCTGCTTTACGCTTTTCT |
| | | GAG (SEQ ID NO: 415) | (SEQ ID NO: 416) |
| NOT | NOTCH1 | CTGCATGCTGGCCTCCG (SEQ ID | CGGTGACTGCTCCCTCAAC (SEQ |
| CH1 | _23 | NO: 417) | ID NO: 418) |
| NOT | NOTCH1 | GGTGAGGAAGGGGTGCT (SEQ | GAGCTTCCTGAGTGGAGAG |
| CH1 | _2 | ID NO: 419) | (SEQ ID NO: 420) |
| NOT | NOTCH1 | GGTCCCGATCCTGTGTCTCC | ACCTGCAGTGTGGTCCC (SEQ ID |
| CH1 | _40 | (SEQ ID NO: 421) | NO: 422) |
| NOT | NOTCH1 | GGTTGTACTCGTCCAGCAG | GTGATGGAACCTTGGGGAG |
| CH1 | _10 | (SEQ ID NO: 423) | (SEQ ID NO: 424) |
| NOT | NOTCH1 | TGACCGTTCCCACCTCCC (SEQ | ACGGCATCAACTCGTTCACC |
| CH1 | _33 | ID NO: 425) | (SEQ ID NO: 426) |
| NOT | NOTCH1 | CGCTTGGCGGCATCAGAG (SEQ | GGACCGGCCCTCCAGA (SEQ ID |
| CH1 | _14 | ID NO: 427) | NO: 428) |
| HRA | HRAS_2 | CCTCCCTCACTGCCCTG (SEQ ID | CGAGACCTCGGCCAAGAC (SEQ |
| S | | NO: 429) | ID NO: 430) |
| PIK3 | PIK3CA_ | GTCCATTGGCATGGGGAAATAT | GCATGGAGTTTCCTAAGAGATG |
| CA | 15 | AA (SEQ ID NO: 431) | GA (SEQ ID NO: 432) |
| CAS | CASP8_8 | CCTTTCCTGCCATGTCTCATTCT | AGTAAGTTTCTGCCCCTTGTCA |
| P8 | | (SEQ ID NO: 433) | G (SEQ ID NO: 434) |
| FAT1 | FAT1_9 | TCGGTCCAGATTGTTTCTTGAG | TTACTGGTGGTGGTGTTTGTTCT |
| | | T (SEQ ID NO: 435) | (SEQ ID NO: 436) |
| PIK3 | PIK3CA_ | CTCGAAGTATGTTGCTATCCTC | ACTCATCCTCAATGTGATTACTT |
| CA | 8 | T (SEQ ID NO: 437) | TTT (SEQ ID NO: 438) |
| NOT | NOTCH1 | TGGCACAGTCATCAATGTTCTC | CATTGCCACCCTGCGTCTTA |
| CH1 | _53 | G (SEQ ID NO: 439) | (SEQ ID NO: 440) |
| NOT | NOTCH1 | AGACTCCCGGTGAGGATGC | CGCCTTTGTGCTTCTGTTCTTC |
| CH1 | _17 | (SEQ ID NO: 441) | (SEQ ID NO: 442) |
| PIK3 | PIK3CA_ | ACATGTCAACCTTTTGAACAGC | TGTGGACTTTCTGAGAGAAAAC |
| CA | 20 | A (SEQ ID NO: 443) | AAT (SEQ ID NO: 444) |
| PIK3 | PIK3CA_ | TGCATCTGTGGCATTAAATGGT | ACATACAGGTTGCCTTACTGGT |
| CA | 31 | G (SEQ ID NO: 445) | T (SEQ ID NO: 446) |
| PIK3 | PIK3CA_ | TCGAGTGTGTGCATATGTGTAT | CTTACCTGGGATTGGAACAAGG |
| CA | 11 | GT (SEQ ID NO: 447) | T (SEQ ID NO: 448) |
| CAS | CASP8_4 | CGCCAGTTAGTCCTAAAAACCC | CCGGACCCTTTTATCTCTAAGCC |
| P8 | | A (SEQ ID NO: 449) | (SEQ ID NO: 450) |
| FAT1 | FAT1_74 | TTCTGTTTCTGGCGCTGTATCTT | TGCCTTTCTCGTATTTTATTTTG |
| | | (SEQ ID NO: 451) | CC (SEQ ID NO: 452) |
| FAT1 | FAT1_14 | TCCTCTACACTGGCAAACAAAG | ACCTCGGAGAATAAAGATAAA |
| | | C (SEQ ID NO: 453) | AAGCA (SEQ ID NO: 454) |
| TP53 | TP53_7 | CACTGACAACCACCCTTAACCC | GGGCTGGAGAGACGACAGG |
| | | (SEQ ID NO: 455) | (SEQ ID NO: 456) |
| FAT1 | FAT1_40 | CAGTTGCCGTGATAGTAAGAAG | TGATTATTATCTTCCTCCATTGA |
| | | (SEQ ID NO: 457) | TTT (SEQ ID NO: 458) |
| FAT1 | FAT1_30 | GGCCGCTACAAAAACTAGATTG | AGTAGAGGCCACTGATGGAGG |
| | | A (SEQ ID NO: 459) | (SEQ ID NO: 460) |
| FAT1 | FAT1_36 | ATAAGGATCGGCAGAGAATTC | TTGAAAGACAAAAGGGGAGGA |
| | | GG (SEQ ID NO: 461) | AA (SEQ ID NO: 462) |
| FAT1 | FAT1_78 | AGGTTTATAGTCACAACATATG | GAAATCTCCTACAGCATCGAAG |
| | | AAAA (SEQ ID NO: 463) | A (SEQ ID NO: 464) |
| FAT1 | FAT1_25 | TGAGGGTCGAGACTGTAGGTTA | ACAGTGCCATAGACAAAGGTGA |
| | | G (SEQ ID NO: 465) | TT (SEQ ID NO: 466) |
| FAT1 | FAT1_86 | GTCATTACCAATGAACCAACAG | ATAAACTCAGTGGAGCAGTTAG |
| | | G (SEQ ID NO: 467) | G (SEQ ID NO: 468) |
| FAT1 | FAT1_43 | TCTCTTTTCATGGTCAAGTTCCT | CCAAGTGAAAGATGCAAATGAC |
| | | (SEQ ID NO: 469) | AAC (SEQ ID NO: 470) |
| FAT1 | FAT1_68 | TGTAACCATCCCAACGAAACTC | TTGATCCTGTCTTGGGCTCTATT |
| | | C (SEQ ID NO: 471) | (SEQ ID NO: 472) |
| FAT1 | FAT1_10 | TATACTCCTTACTCCCTGGAAA | CCAAGCTAACGGTGCACAT |
| | 1 | G (SEQ ID NO: 473) | (SEQ ID NO: 474) |
| FAT1 | FAT1_62 | AAATCTGCCATCGGAAGCTCTA | TTTGCCAAGCCATTATATGAAG |
| | | A (SEQ ID NO: 475) | C (SEQ ID NO: 476) |
| FAT1 | FAT1_95 | CGATCGCTTTAATGACAATACC | CGGGAAGTCGAAGTCCTTG |
| | | C (SEQ ID NO: 477) | (SEQ ID NO: 478) |
| FAT1 | FAT1_54 | GTCCACCGTGACAATCACATCA | TGGAACGTCTGTTGTTCAAGTT |
| | | (SEQ ID NO: 479) | A (SEQ ID NO: 480) |
| FAT1 | FAT1_83 | ACTTTTCCCCCTAACAGAAAAG | GTCAATGACAATGCACCACAGA |
| | | CA (SEQ ID NO: 481) | C (SEQ ID NO: 482) |
| CAS | CASP8_3 | CTACATTCCGCAAAGGAAGCA | GTCCCACAATGGGAGTTTCCAC |
| P8 | | AG (SEQ ID NO: 483) | (SEQ ID NO: 484) |
| CAS | CASP8_1 | GTTGAGACTGATTCAGAGGAGC | GTAACAGTGAGGAGGGCTGAGT |
| P8 | 4 | A (SEQ ID NO: 485) | A (SEQ ID NO: 486) |
| NOT | NOTCH1 | AGAGGGTTGTATTGGTTCGG | GGCCAAGCCCGAGATG (SEQ ID |
| CH1 | _6 | (SEQ ID NO: 487) | NO: 488) |
| NOT | NOTCH1 | CAGGTGGGCAGCACCAAA (SEQ | CTTCAGGACCCAACTGCGAGAT |
| CH1 | _43 | ID NO: 489) | (SEQ ID NO: 490) |
| NOT | NOTCH1 | CGTGGACCTCTCCAGGTGT | GCGTGTGGGGCAGCAG (SEQ ID |
| CH1 | _27 | (SEQ ID NO: 491) | NO: 492) |
| HRA | HRAS_4 | GACATGCGCAGAGAGGACAG | GAGACGTGCCTGTTGGACATC |
| S | | (SEQ ID NO: 493) | (SEQ ID NO: 494) |
| PIK3 | PIK3CA_ | TCCATGCTTAGAGTTGGAGTTT | CCAGTAAAATATATGGATCCTT |
| CA | 17 | G (SEQ ID NO: 495) | TTCC (SEQ ID NO: 496) |
| PIK3 | PIK3CA_ | AATTGGGGAAAGGCAGTAAAG | CGCCTTTGCACTGAATTTGCAT |
| CA | 32 | GT (SEQ ID NO: 497) | (SEQ ID NO: 498) |
| PIK3 | PIK3CA_ | TCCTTCTTTGATTTAGGTTTCTG | ATGGTTATTAATGTAGCCTCAC |
| CA | 1 | C (SEQ ID NO: 499) | G (SEQ ID NO: 500) |

Sequencing probes useful for detecting HPV. screening for HPV. or determining the presence of an HPV injection may include one or more probes provided in the Table below:

| Gene/Chr | Assay ID | F-sp | R-sp |
|---|---|---|---|
| HPV | TXA04 | ATGGGTGTATTTTTTGGTGGGTT | GGGGAATGGAAGGTACAGATGT |
| 16 | 06966 | (SEQ ID NO: 501) | T (SEQ ID NO: 502) |
| HPV | TXA04 | AGATTGGTGGTGTTTACATTTCC | TGGTCACGTAGGTCTGTACTATC |
| 16 | 06995 | T (SEQ ID NO: 503) | (SEQ ID NO: 504) |
| HPV | TXA04 | GTAGTAGAAAGCTCAGCAGACG | AATCTACCATATCCGACCCTGTG |
| 18 | 07021 | A (SEQ ID NO: 505) | (SEQ ID NO: 506) |
| HPV | TXA04 | ATGAATATGTTGCACGCACAAA | CCTACCTCAACACCTACACAGG |
| 16 | 07028 | C (SEQ ID NO: 507) | (SEQ ID NO: 508) |
| HPV | TXA04 | AAAAAGGGAGTAACCGAAAACG | CACAGGTTATTTCTATGTCTTGC |
| 18 | 07031 | G (SEQ ID NO: 509) | AGT (SEQ ID NO: 510) |
| HPV | TXA04 | TGCAGTACAGGTTCTAAAACGA | ACATGGTGTTTCAGTCTCATGGC |
| 16 | 07048 | A (SEQ ID NO: 511) | (SEQ ID NO: 512) |
| nc | TXA04 | CCAGAAAGTTACCACAGTTATG | ATTGCTGTTCTAATGTTGTTCCA |
| | 07053 | C (SEQ ID NO: 513) | (SEQ ID NO: 514) |
| nc | TXA04 | CATTTAACAGCTCACACAAAGG | TTGAGACAAAAATTGGTCACGT |
| | 07061 | A (SEQ ID NO: 515) | T (SEQ ID NO: 516) |
| nc | TXA04 | CGGACAGAGCCCATTACAATA | TATGGTTTCTGAGAACAGATGG |
| | 07063 | (SEQ ID NO: 517) | G (SEQ ID NO: 518) |
| HPV | TXA04 | TTGGAGGACTGGAATTTTGGTCT | TTTTGGTTTGGCCTTCAATCCTG |
| 16 | 07069 | (SEQ ID NO: 519) | (SEQ ID NO: 520) |
| nc | TXA04 | GAATTCGGTTGCATGCTTTTTG | CGTTGGCGCATAGTGATTTATTT |
| | 07077 | (SEQ ID NO: 521) | (SEQ ID NO: 522) |
| HPV | TXA04 | ACTTTAACTGCAGATGTTATGTC | AACTTTTCCTTTAAATCCACATT |
| 18 | 07108 | CT (SEQ ID NO: 523) | CCA (SEQ ID NO: 524) |
| HPV | TXA04 | TGAACTGCAAATGGCCCTACAA | GTTTTGTCCCATGTTCCTGCATC |
| 18 | 07113 | G (SEQ ID NO: 525) | (SEQ ID NO: 526) |
| HPV | TXA04 | TCATGCTGGCAGCTCTAGATTAT | ACTAAGGCCAACACCTAAAGGC |
| 18 | 07114 | T (SEQ ID NO: 527) | (SEQ ID NO: 528) |
| nc | TXA04 | AATATTGGTGGGATACATGACA | TGCAATAGTAACATGGGCAATA |
| | 07115 | A (SEQ ID NO: 529) | A (SEQ ID NO: 530) |
| HPV | TXA04 | GGGCACTGCTCCTACATATTTTG | GGGTAGACAGAATGTTGGACAT |
| 18 | 06951 | A (SEQ ID NO: 531) | GA (SEQ ID NO: 532) |
| HPV | TXA04 | CTTTAGTATCTGCCACGGAGGAC | GGTGATACAATGGGCCATACAG |
| 18 | 07025 | (SEQ ID NO: 533) | A (SEQ ID NO: 534) |
| HPV | TXA04 | GAGGTCCACAATGATGCACAAG | TACCTGAATCTGTGTTGCTTCCA |
| 18 | 07026 | T (SEQ ID NO: 535) | (SEQ ID NO: 536) |
| HPV | TXA04 | GCAGCTGTGTGTATTCTCCCT | CCTCAACATGTCTGCTATACTGC |
| 18 | 07035 | (SEQ ID NO: 537) | TT (SEQ ID NO: 538) |
| nc | TXA04 | ACATTTACCTGACCCCAATAAGT | CAATTGTGTTTGTTTGTAATCCA |
| | 07045 | (SEQ ID NO: 539) | T (SEQ ID NO: 540) |
| HPV | TXA04 | ACACCTACTAATTGTGTTGTGGT | AGGTCAGGAAAACAGGGATTTG |
| 16 | 07049 | T (SEQ ID NO: 541) | G (SEQ ID NO: 542) |
| HPV | TXA04 | CCCTTTAACAGTAGATCCTGTGG | TGTTGGAGGCTGCAATACAGAT |
| 16 | 07070 | G (SEQ ID NO: 543) | (SEQ ID NO: 544) |
| HPV | TXA04 | TTGTGGACCAGCAAATACAGGA | AGTTGTATTAATGGTTTGTGCTT |
| 18 | 07078 | (SEQ ID NO: 545) | TCT (SEQ ID NO: 546) |
| HPV | TXA04 | TGGAGACACATTGGAAAAACTA | TGTTGCCTTAGGTCCATGCATAC |
| 18 | 07095 | ACT (SEQ ID NO: 547) | (SEQ ID NO: 548) |
| HPV | TXA04 | TGGAGACTCTTTGCCAACGTTTA | GCAGTTCAATTGCTTGTAATGCT |
| 16 | 07096 | (SEQ ID NO: 549) | (SEQ ID NO: 550) |
| HPV | TXA04 | ATACATTAAAGGCTCTGGGTCTA | TTCTGAAGTAGATATGGCAGCA |
| 16 | 07099 | C (SEQ ID NO: 551) | C (SEQ ID NO: 552) |
| HPV | TXA04 | CAGACACCGGAAACCCCTG (SEQ | TACATTATGTCCTGTCCAATGCC |
| 16 | 07103 | ID NO: 553) | (SEQ ID NO: 554) |
| HPV | TXA04 | TGGCAATACTGAAGTGGAAACT | TTTGCTAACATTGCTGCCTTTGC |
| 16 | 07104 | C (SEQ ID NO: 555) | (SEQ ID NO: 556) |
| HPV | TXA04 | GGTACATGTCCACCTGATGTTGT | TGTAACAATAGATGGGTCTGTG |
| 18 | 07107 | (SEQ ID NO: 557) | GG (SEQ ID NO: 558) |
| HPV | TXA04 | AACGTTCTGCTCCATCTGC (SEQ | ACCACATAACACACAGAACCAC |
| 18 | 07029 | ID NO: 559) | A (SEQ ID NO: 560) |
| nc | TXA04 | CTGCTTTTGTCTGTGTCTACAT | ACATTATGTAATTAAAAAGCGT |
| | 07039 | (SEQ ID NO: 561) | GCAT (SEQ ID NO: 562) |
| HPV | TXA04 | ACTGGCATTAGGTACAGTAGAA | CCGGGGTTGTAGAAGTATCTGT |
| 16 | 07042 | TTGG (SEQ ID NO: 563) | AAT (SEQ ID NO: 564) |
| HPV | TXA04 | AAATAAATCACTATGCGCCAAC | AGTTGCTTGTAAATGTGTAACCC |
| 16 | 07044 | G (SEQ ID NO: 565) | AAA (SEQ ID NO: 566) |
| nc | TXA04 | AATTGGATGACACAGAAAATGC | ATAGCACCAAAGCCAGTATCAA |
| | 07059 | T (SEQ ID NO: 567) | (SEQ ID NO: 568) |
| nc | TXA04 | TTTAAGGAGTACCTACGACATG | GCTGGAGGTGTATGTTTTTGAC |
| | 07064 | G (SEQ ID NO: 569) | (SEQ ID NO: 570) |
| nc | TXA04 | GTCAGTACAGTAGTGGAAGTGG | GTAAGTCCAAATGCAGCAATAC |
| | 07065 | (SEQ ID NO: 571) | A (SEQ ID NO: 572) |
| HPV | TXA04 | AGGTGATTGGAAGCAAATTGTT | ACCTATTTTGGCATCTGCTAATG |
| 16 | 07066 | ATGT (SEQ ID NO: 573) | GT (SEQ ID NO: 574) |
| HPV | TXA04 | ATTAGGTCAACGGGCAACTATG | GGCAGAAGATATAGTGGGCGAA |
| 18 | 07083 | T (SEQ ID NO: 575) | (SEQ ID NO: 576) |
| HPV | TXA04 | ACTGAACACTTCACTGCAAGAC | ACCCAGTGTTAGTTAGTTTTTCC |
| 18 | 07086 | A (SEQ ID NO: 577) | A (SEQ ID NO: 578) |
| HPV | TXA04 | TGTCCTCCAATACTACTAACCAC | TCTAATCTGGACCATGTCCTTTC |
| 18 | 07087 | A (SEQ ID NO: 579) | A (SEQ ID NO: 580) |
| HPV | TXA04 | TGGGCAGAAAAAGGCAAAAAG | ATTGTGCTATGGTACATTGTGGA |
| 18 | 07091 | G (SEQ ID NO: 581) | (SEQ ID NO: 582) |
| HPV | TXA04 | CGATTGCCCCCCTTTAGAACTTA | ACCTGCTCTATTCCAAAAATGCC |
| 18 | 07092 | (SEQ ID NO: 583) | (SEQ ID NO: 584) |
| HPV | TXA04 | GTGCAGGCAATGAAAAAACAGG | ATGCATACGCACACATACAGAC |
| 18 | 07093 | A (SEQ ID NO: 585) | A (SEQ ID NO: 586) |
| HPV | TXA04 | TGTATTTAACTGCACCAACAGG | TCTGCATCATCTTTAAACTGCAC |
| 16 | 06164 | A (SEQ ID NO: 587) | A (SEQ ID NO: 588) |
| HPV | TXA04 | TGTGAAAAATATGGGAACACAG | AGGTCCACAATGCTGCTTCTC |
| 18 | 07022 | GT (SEQ ID NO: 589) | (SEQ ID NO: 590) |
| nc | TXA04 | CATATGGCGACAGCTTATTTTTT | ATTATTGTGGCCCTGTGCT (SEQ |
| | 07038 | (SEQ ID NO: 591) | ID NO: 592) |
| nc | TXA04 | TGCTGTCTAAACTATTATGTGTG | CTATGTCATTATCGTAGGCCCAT |
| | 07054 | TC (SEQ ID NO: 593) | (SEQ ID NO: 594) |
| HPV | TXA04 | AAACTAAGGGCGTAACCGAAAT | TGCATAAATCCCGAAAAGCAAA |
| 16 | 07058 | C (SEQ ID NO: 595) | GT (SEQ ID NO: 596) |
| HPV | TXA04 | TACTGCATCCACAACATTACTGG | GTAATTAAAAAGCGTGCATGTG |
| 16 | 07067 | (SEQ ID NO: 597) | TATG (SEQ ID NO: 598) |
| HPV | TXA04 | TGGAGTAAACCCAACAATAGCA | TGCTGCAACACTACTTCGCAAT |
| 18 | 07079 | G (SEQ ID NO: 599) | (SEQ ID NO: 600) |
| HPV | TXA04 | AACATTTGCTTCTTCTGGTACGG | TCAGGAACATCACTACGAGGAT |
| 18 | 07080 | (SEQ ID NO: 601) | CA (SEQ ID NO: 602) |
| HPV | TXA04 | ATGTCCTGTGTTTGTGTTTGTTG | AAAATGGATGCTGTAAGGTGTG |
| 18 | 07081 | (SEQ ID NO: 603) | C (SEQ ID NO: 604) |
| HPV | TXA04 | ACCACGTGTTGGACATACTTTGA | TGGCCATCTATTATCCTTTGCTG |
| 18 | 07082 | (SEQ ID NO: 605) | (SEQ ID NO: 606) |
| HPV | TXA04 | TCCTTATGGGGATTCCATGTTTT | GACCCTGTGCCTTATGTAACCAA |
| 18 | 07085 | (SEQ ID NO: 607) | (SEQ ID NO: 608) |
| HPV | TXA04 | AGAGGGTACGGGATGTAATGGA | TCGTTTTAGAACCTGTACTGCAT |
| 16 | 07097 | T (SEQ ID NO: 609) | (SEQ ID NO: 610) |
| HPV | TXA04 | TGTTAGCACAAACCCTAACACA | TCCAAAAAGTCAGGATCTGGAG |
| 16 | 07100 | G (SEQ ID NO: 611) | C (SEQ ID NO: 612) |
| HPV | TXA04 | TGCGACGTGAGGTATATGACTTT | TTCTTCAGGACACAGTGGCTTTT |
| 16 | 06987 | (SEQ ID NO: 613) | (SEQ ID NO: 614) |
| HPV | TXA04 | CCCCACAGACCCATCTATTGTTA | CACCTCCCCAGTTTGTGGAAC |
| 18 | 07037 | (SEQ ID NO: 615) | (SEQ ID NO: 616) |
| HPV | TXA04 | TTTGTAACCACTCCCACTAAACT | ACCTTAGCACCTATAGATTTTCC |
| 16 | 07043 | (SEQ ID NO: 617) | ACT (SEQ ID NO: 618) |
| HPV | TXA04 | TGGCAGACACTAATAGTAATGC | TGACATAAACTCTACACCTTGAT |
| 16 | 07052 | AAGT (SEQ ID NO: 619) | ACC (SEQ ID NO: 620) |
| HPV | TXA04 | CTACATGGCATTGGACAGGACA | ATAGGCAGACACACAAAAGCAC |
| 16 | 07072 | T (SEQ ID NO: 621) | A (SEQ ID NO: 622) |
| HPV | TXA04 | GGTACCAATGGGGAAGAGGGTA | CTGTCTCTGTTTCTGCCTGTGTT |
| 16 | 07073 | (SEQ ID NO: 623) | (SEQ ID NO: 624) |
| HPV | TXA04 | AACCAACTATTTGTTACTGTTGT | GGGAGGTTGTAGACCAAAATTC |
| 16 | 07075 | TGA (SEQ ID NO: 625) | CA (SEQ ID NO: 626) |
| HPV | TXA04 | AGCAATTAAGCGACTCAGAGGA | GGATCAGCCATTGTTGCTTACTG |
| 18 | 07094 | A (SEQ ID NO: 627) | (SEQ ID NO: 628) |
| HPV | TXA04 | TATTACAAGGCCAGAGAAATGG | CTCCATCAAACTGCACTTCCACT |
| 16 | 07098 | G (SEQ ID NO: 629) | (SEQ ID NO: 630) |
| HPV | TXA04 | TTAACCTCCTCTTGGGATGTGC | AGAAGGAGGTGGAAGATATACG |
| 18 | 07109 | (SEQ ID NO: 631) | G (SEQ ID NO: 632) |
| HPV | TXA04 | GGTAGATACCACTCCCAGTACC | GGGAACACCAAAGTTCCAATCC |
| 18 | 07111 | A (SEQ ID NO: 633) | T (SEQ ID NO: 634) |
| HPV | TXA04 | TGACAGTAAAGACATAGACAGC | TTCCATAGTTCCTCGCATGTGTC |
| 18 | 07112 | CAA (SEQ ID NO: 635) | (SEQ ID NO: 636) |
| HPV | TXA04 | ATGGTGCAGCTAACACAGGTAA | ACCAATGGTCTATGCTTTACATC |
| 16 | 07005 | A (SEQ ID NO: 637) | CA (SEQ ID NO: 638) |
| HPV | TXA04 | ACCTCTGTGTCTATTTCCACAAC | CTACTGTAAAGGCGGGGACCTG |
| 18 | 07023 | C (SEQ ID NO: 639) | (SEQ ID NO: 640) |
| HPV | TXA04 | GAGAAGCAGCATTGTGGACCT | TCCTGTTTTTTCATTGCCTGCAC |
| 18 | 07024 | (SEQ ID NO: 641) | (SEQ ID NO: 642) |
| HPV | TXA04 | GTTTGTGGTATGGGTGTTGCTTG | GCGCCAATTGTTCAAAATATGTA |
| 18 | 07033 | (SEQ ID NO: 643) | GG (SEQ ID NO: 644) |
| HPV | TXA04 | TTCTGCAAAACGCACAAAACG | GCAAGTGTATCTGTAGCTGTGG |
| 16 | 07040 | (SEQ ID NO: 645) | G (SEQ ID NO: 646) |
| HPV | TXA04 | ACCGGTCGATGTATGTCTTGT | AGTCACACTTGCAACAAAAGGT |
| 16 | 07041 | (SEQ ID NO: 647) | T (SEQ ID NO: 648) |
| HPV | TXA04 | TGGCTTTGGTGCTATGGACTTTA | TTGCAGTAGACCCAGAGCCTTTA |
| 16 | 07047 | (SEQ ID NO: 649) | (SEQ ID NO: 650) |
| HPV | TXA04 | ACATCTGTGTTTAGCAGCAACG | TAAATGCAGTGAGGATTGGAGC |
| 16 | 07050 | A (SEQ ID NO: 651) | A (SEQ ID NO: 652) |
| HPV | TXA04 | TTCACATGCAGCCTCACCTACTT | TGGAGACCCTGGAACTATAGGA |
| 16 | 07076 | (SEQ ID NO: 653) | A (SEQ ID NO: 654) |
| HPV | TXA04 | AGAAATGGTACAATGGGCATTT | GTCTCCAATCTCCCCCTTCATCT |
| 18 | 07089 | GAT (SEQ ID NO: 655) | (SEQ ID NO: 656) |
| HPV | TXA04 | AACTAGTAATGCAAAGGCAGCA | AACCATTCCCCATGAACATGCTA |
| 16 | 07102 | A (SEQ ID NO: 657) | (SEQ ID NO: 658) |
| HPV | TXA04 | TACAAGCAGGATTGAAGGCCAA | CAAGCATTTAAAAACACATACA |
| 16 | 07105 | A (SEQ ID NO: 659) | CACG (SEQ ID NO: 660) |
| HPV | TXA04 | GACTCCAACGACGCAGAGAAA | CAACATTGTGTGACGTTGTGGTT |
| 18 | 07110 | (SEQ ID NO: 661) | (SEQ ID NO: 662) |
| HPV | TXA04 | TGCCAAATCCCTGTTTTCCTGA | AGGGTGACATTTAGTTGGCCTTA |
| 16 | 06988 | (SEQ ID NO: 663) | (SEQ ID NO: 664) |
| HPV | TXA04 | TAGAACAGCAATACAACAAACC | TCATGCAATGTAGGTGTATCTCC |
| 16 | 06994 | G (SEQ ID NO: 665) | (SEQ ID NO: 666) |
| nc | TXA04 | TCCTGCAAATACAACAATTCCTT | CAGCCAAAGAGACATCTGAAAA |
| | 07018 | (SEQ ID NO: 667) | A (SEQ ID NO: 668) |
| HPV | TXA04 | CCAAAATTGCGAAGTAGTGTTG | GCTGCATTGCTGTTGCTGTC |
| 18 | 07019 | C (SEQ ID NO: 669) | (SEQ ID NO: 670) |
| HPV | TXA04 | CAGCCTTTAGGTGTTGGCCTTA | GCAATCGCCCTGTGATAAAGGA |
| 18 | 07020 | (SEQ ID NO: 671) | (SEQ ID NO: 672) |
| nc | TXA04 | TTGCTTGTCAAAAACATACACCT | TACAGCTTACGTTTTTTGCGTTT |
| | 07046 | (SEQ ID NO: 673) | (SEQ ID NO: 674) |
| HPV | TXA04 | GCAAAGGATCCCCATGTACCAA | TCCAGTGGAACTTCACTTTTGTT |
| 16 | 07062 | T (SEQ ID NO: 675) | (SEQ ID NO: 676) |
| HPV | TXA04 | ATACATACACATGCACGCTTTTT | GTACCTGCCTGTTTGCATGTTTT |
| 16 | 07074 | (SEQ ID NO: 677) | (SEQ ID NO: 678) |
| HPV | TXA04 | ATGTGTGCGTATGCATGGGTATT | GCACGGTGGGATACCATACTTTT |
| 18 | 07084 | (SEQ ID NO: 679) | (SEQ ID NO: 680) |
| HPV | TXA04 | ACGGCTGGTTTTATGTACAAGCT | GACTGTTTTCTGTGCTGCCTCC |
| 18 | 07090 | A (SEQ ID NO: 681) | (SEQ ID NO: 682) |
| HPV | TXA04 | TATGGAGACACGCCAGAATGGA | TGCACAATCCTTTACAATTTTTG |
| 16 | 07101 | T (SEQ ID NO: 683) | CC (SEQ ID NO: 684) |
| HPV | TXA04 | AATGAATATGTCACAGTGGATA | ATGATTTTCCTGTATTTGCTGGT |
| 18 | 07027 | CGA (SEQ ID NO: 685) | C (SEQ ID NO: 686) |
| HPV | TXA04 | AAGTAATAAAACTGCTTTTAGG | AGTATGTGCTGCCCAACCTATTT |
| 18 | 07030 | CACA (SEQ ID NO: 687) | (SEQ ID NO: 688) |
| HPV | TXA04 | CAACAACAGCAGTGTAGACGGT | ACTCCAAATATAGCTGTAACCC |
| 18 | 07032 | A (SEQ ID NO: 689) | AAT (SEQ ID NO: 690) |
| HPV | TXA04 | AACGTAAACGTGTTCCCTATTTT | GCAGAAACCTTAGGAATATCCT |
| 18 | 07034 | TT (SEQ ID NO: 691) | GCT (SEQ ID NO: 692) |
| HPV | TXA04 | GATGCAGGAACATGGGACAAAA | GCTGTTTAACAAGCTGAGTAGC |
| 18 | 07036 | C (SEQ ID NO: 693) | G (SEQ ID NO: 694) |
| nc | TXA04 | TATTGCTGCATTTGGACTTACAC | CAATTTTGGAGGCTCTATCATCA |
| | 07051 | (SEQ ID NO: 695) | (SEQ ID NO: 696) |
| HPV | TXA04 | TTGTATGTGCTTGTATGTGCTTG | GCCGCTGGCGCTACAAAATA |
| 16 | 07055 | T (SEQ ID NO: 697) | (SEQ ID NO: 698) |
| HPV | TXA04 | ACATGAACTGTGTAAAGGTTAG | AACTTTCTGGGTCGCTCCTG |
| 16 | 07056 | TCAT (SEQ ID NO: 699) | (SEQ ID NO: 700) |
| HPV | TXA04 | GTAACTGTGGTAGAGGGTCAAG | CGACGGCTTTGGTATGGGTC |
| 16 | 07057 | T (SEQ ID NO: 701) | (SEQ ID NO: 702) |
| HPV | TXA04 | AGTTCCAGGGTCTCCACAATATA | CTGCAAGTAGTCTGGATGTTCCT |
| 16 | 07060 | C (SEQ ID NO: 703) | (SEQ ID NO: 704) |
| HPV | TXA04 | CAACTGATACCACACCTGCT | GGGCGAGACCCTGGTA (SEQ ID |
| 16 | 07068 | (SEQ ID NO: 705) | NO: 706) |
| HPV | TXA04 | CAGTGCCCTGTTGGAACTACATA | AGCTCATACACTGGATTTCCGTT |
| 16 | 07071 | (SEQ ID NO: 707) | (SEQ ID NO: 708) |
| HPV | TXA04 | TGCTATTACCTGTCAAAAGGATG | ACGTACACGCACACGCTT (SEQ |
| 18 | 07088 | C (SEQ ID NO: 709) | ID NO: 710) |
| HPV | TXA04 | ATTACATATGACAACCCGGCCTT | ATTCTGGGGAAGGTGCAATAGG |
| 18 | 07106 | (SEQ ID NO: 711) | A (SEQ ID NO: 712) |

### IX. Detection Methods

Some embodiments of the disclosure relate to methods of detecting a HNC in a subject. The methods generally comprise a single assay which performs two functions. First. the assay detects somatic mutations. These mutations are linked to the presence of a HNC. In some embodiments, these mutations are indicative of OCSCC. Second, the assay also detects the presence or absence of human papilloma virus (HPV).

The mutations detected by the assay are NOTCH1, TP53, FAT1, PIK3CA, CDKN2A, HRAS, or CASP8. While each of these mutations in queried by the assay, the presence of any of these mutations is generally indicative of cancer. Accordingly, the assay may detect one or more than one of the mutations in a single sample.

The assay also detects the presence or absence of HPV. This detection method may be used in combination with the mutation detection or independently to provide the HPV status of a subject. In some embodiments, the assay detects HPV 16 and/or HPV 18.

The method assays a sample. The sample is generally derived from the mouth of the subject (i.e., an oral sample). In some embodiments, the oral sample comprises saliva from the subject. In some embodiments, the oral sample comprises an oral rinse sample from the subject.

Regardless, the oral samples are evaluated for the presence or absence of certain genetic material or DNA sequences. In some embodiments, the DNA sequences relate to the somatic mutations identified above. In some embodiments, the DNA sequences relate to HPV. Accordingly, in some embodiments, the oral sample comprises genetic material from the oral cavity. Further, in some embodiments, the oral sample comprises cancer-specific or viral-specific DNA.

As identified above, detection of the mutations or viral sequences comprise sequencing the genetic material of the oral sample. In some embodiments, the sequencing comprises next generation sequencing (NGS).

Some methods of this disclosure also provide an indication of a type of HNC. The presence of HPV is indicative of OPSCC, while the absence of HPV is indicative of OCSCC. Accordingly, the presence of HPV indicates that the subject has or is at high risk of having OPSCC. Similarly, the absence of HPV indicates that the subject has or is at low risk of having OPSCC. This “typing” of a HNC can be helpful to a practitioner in locating a HNC without invasive procedures (e.g., biopsies).

### X. HPV Detection

Some embodiments of this disclosure provide methods of detecting HPV in an oral sample. The sample is, as above, any sample derived from the oral cavity of a subject.

### XI. Examples

The examples provided herein and in the Appendices are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Head and neck cancers (HNC) consist of a group of biologically and clinically diverse malignancies arising from the oral cavity, pharynx, larynx, paranasal sinuses, nasal cavity, and salivary glands. Oral cavity squamous cell carcinoma (OCSCC) and oropharyngeal squamous cell carcinoma (OPSCC) are the most common subtypes, together comprising the majority of HNC cases. While these cancers are associated with tobacco use and alcohol consumption, infection with human papilloma virus (HPV) is also etiologic to HNC, with OPSCC known to harbor higher HPV positivity rates relative to OCSCC. which is largely HPV− negative (HPV−). This distinction is clinically relevant as HPV− positive (HPV+) tumors are more responsive to therapy and associated with a better prognosis. While early-stage OCSCC/OPSCC has favorable 5-year relative survival rates of >80%, diagnosis typically occurs at more advanced disease stages, where the survival rate drops precipitously to ˜20-40%. Compounding this grim outlook is the growing incidence of HNC in patients that do not smoke or drink alcohol, as well as the startling rise in HPV+ OPSCC incidence, thus increasing the affected population and burden on the healthcare system. Currently, painful incisional biopsies are the standard clinical method to diagnose HNC, and there are no accepted non-invasive screening options available for early disease detection or serial assessment of treatment efficacy. There is thus an urgent need to develop non-invasive diagnostic solutions that accurately identify disease at an early stage, as well as to develop faithful methods to quickly discern HPV status to inform effective treatment decisions and improve patient outcomes.

In one aspect. the invention provides a novel salivary liquid biopsy screening method for early detection of OCSCC, which relies on targeted Next Generation Sequencing (NGS) of 7 commonly mutated genes associated with OCSCC tumorigenesis. Demonstrating the utility of this approach, the inventors have shown that it is able to accurately and reproducibly identify ˜93% of patients with OCSCC, including early stage cases. To enhance the capabilities of this screening platform the inventors have incorporated probes targeting high-risk HPV strains (HPV 16/18) into the sequencing panel. Applying this multi-functional assay to a cohort of primary OCSCC tumors, driving somatic mutations were detected in all cases. Furthermore, using the 5% alignment cutoff, all OCSCC specimens were HPV−. The inventors next sequenced HPV+ OPSCCs using the same criteria, detecting 93% of the cases as HPV positive by the combined assay. The inventors next applied this updated assay to saliva specimens collected from 30 OCSCC patients and 10 healthy individuals. Somatic mutations were detected in all OCSCC saliva specimens, while no actionable aberrations were found in healthy controls. As expected, most of the OCSCC saliva samples (27 of 30) were HPV−, with the remaining 3 samples being inconclusive.

### Example 2

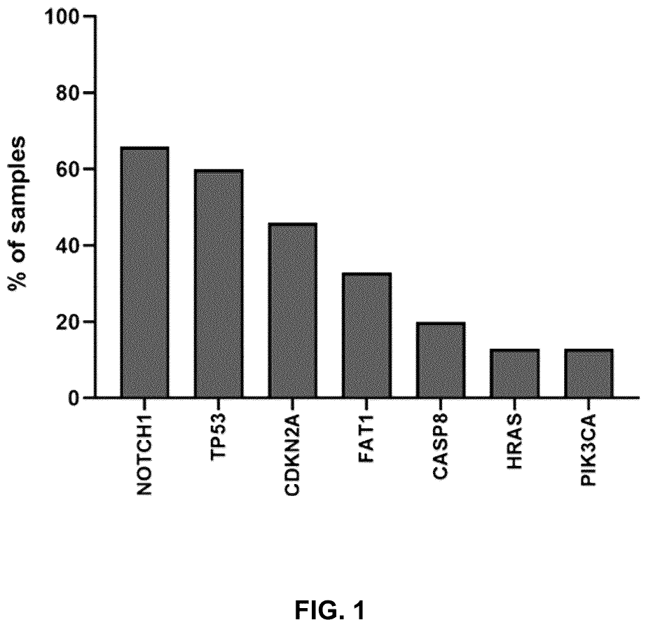

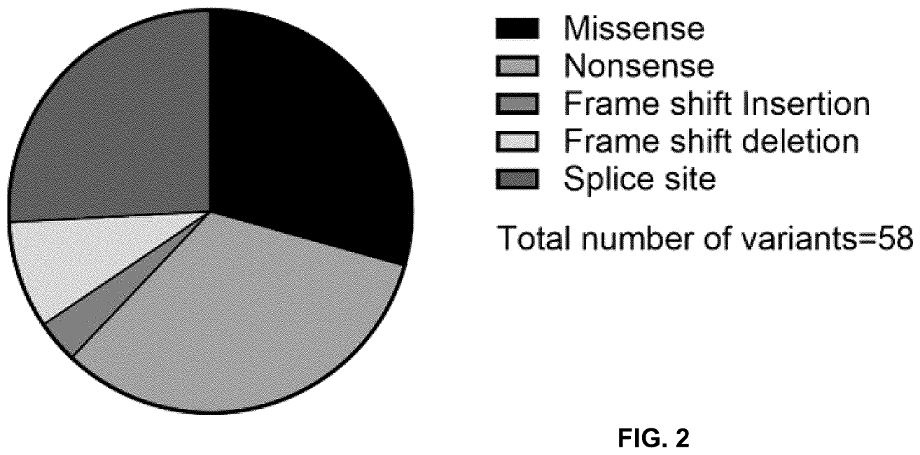

A dual assay for mutation detection in 15 FFPE samples from OSCC patients was tested. Variants in NOTCH1 were identified in 10/15 (66%) cases, TP53 in 9/15 (60%), FAT1 in 7/15 (46%), PIK3CA in 5/15 (33%), CDKN2A in 3/15, HRAS in 2/15 (13%) and CASP8 in 2/15 (13%) cases (see FIG. 1). A total of 58 variants were identified across the 15 samples and majority of variants were missense and nonsense variants followed by splice-site and frame-shift insertion and deletions (see FIG. 2).

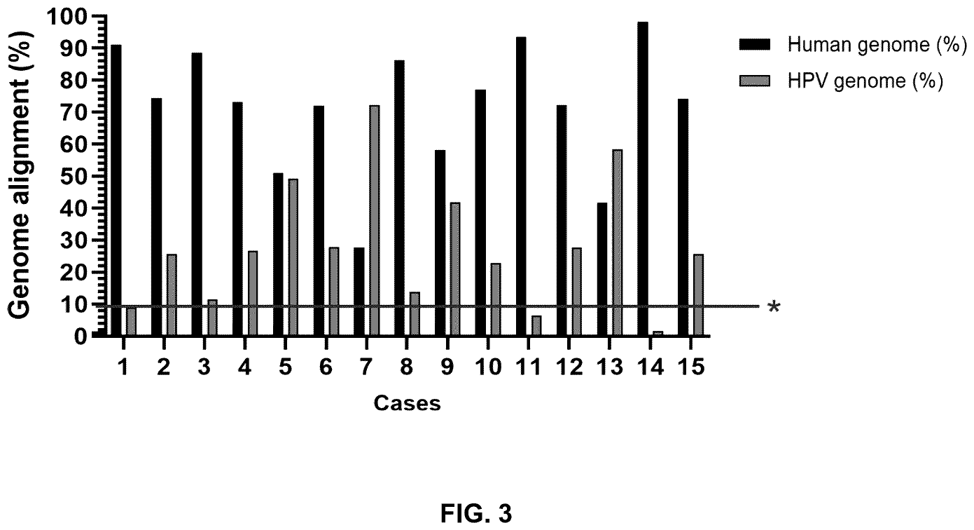

To further validate the assay for HPV detection, 15 FFPE samples from HPV driven oropharyngeal squamous cell carcinoma (OPSCC) were sequenced. The assay was able to detect HPV in 13/15 samples (see FIG. 3). The cutoff for HPV detection in FFPE samples was set at 9% (meaning that any sample with alignment of 9% or more with the HPV genome will be considered as HPV positive). Interestingly, one of the two samples that tested HPV negative on the assay turned out to be HPV33 positive when tested with CLIA certified clinical HPV PCR assay. This could explain the negative result through the assay, which is designed to detect the most prevalent high-risk HPV 16 & 18 strains. Also tested were 44 saliva samples from oral cancer patients and identified HPV in 2/44 (4.5%) samples based on HPV alignment cutoff of 9%. This is consistent with the reports in the literature that suggest HPV positivity of 4-5% in OSCC patients.

### Example 3

The DNA from the saliva samples were extracted using Qiagen QIAamp DNA Blood mini kit and concentration was determined using QUBIT 2.0 Fluorometer. The DNA was then used to prepare sequencing libraries by enriching/amplifying for the 7 genes as well as for HPV 16 & 18 included in the panel using the designed primer pool. The sample libraries were then sequenced on NovaSeqX to generate ˜40-50 million reads per sample. Post sequencing, the sequencing quality was checked for adapter contamination, read length etc. Once the samples pass the quality check, variants are called at variant allele frequency (VAF) of 0.3% for mutation detection. Germline and passenger mutations are filtered out using a proprietary computational approach. The cutoff for HPV detection in saliva was set at 1%. alignment to the HPV reference genome. The cut-off for mutation detection in saliva samples is 0.3% and for HPV detection, the cut-off is 1%.

Design of the positive controls (gBlocks): Synthetic oligonucleotides called gBlocks were designed and used as positive controls for assays to detect cancer-associated mutations. These gBlocks contain known variants at specific locations. The gBlocks were diluted at 1%, 0.5%. 0.2% variant allele frequency and sequenced with our assay. The gBlock samples generated greater than about 10M reads with an average coverage greater than about 90000X. An alignment of greater than about 99% and greater than about 90% on-target reads were observed. Further, the assay was able to detect the known variants in the gBlocks at VAF closer to the expected LOD range. The details on each gblock variant, the supporting reads (%) for the desired VAF is summarized in the table below.

| Chromo- | | | | | Variant | | | |
|---|---|---|---|---|---|---|---|---|
| some | Start | End | Reference | | Allele | Variant Type | | Gene |
| chr2 | 201271529 | 201271529 | C | | T | Substitution | | CASP8 |
| chr2 | 201284951 | 201284955 | GTATC | | G | Deletion | | CASP8 |
| chr2 | 201284960 | 201284965 | CCCATG | | C | Deletion | | CASP8 |
| chr4 | 186617917 | 186617917 | G | | C | Substitution | | FAT1 |
| chr9 | 21971048 | 21971059 | AGCCGCGCCCCG | | A | Deletion | | CDKN2A |
| | | | 0.20% | | 0.50% | | 1% | |
| | | | Support- | | Support- | | Support- | |
| | | | ing | Total | ing | Total | ing | Total |
| | | | Reads % | Reads | Reads % | Reads | Reads % | Reads |
| | | | (EI-VM- | (EI-VM- | (EI-VM- | (EI-VM- | (EI-VM- | (EI-VM- |
| | | | 3s-G- | 3s-G- | 3s-G- | 3s-G- | 3s-G- | 3s-G- |
| | | | block- | block- | block- | block- | Block- | Block- |
| Chromo- | | | 02-per- | 02-per- | 05-per- | 05-per- | 1-per- | 1-per- |
| some | Start | End | cent_S39) | cent_S39) | cent_S38) | cent_S38) | cent_S37) | cent_S37) |
| chr2 | 201271529 | 201271529 | 0.12 | 124462 | 0.09 | 95510 | 0.96 | 178773 |
| chr2 | 201284951 | 201284955 | 0.09 | 198333 | 0.26 | 131220 | 0.86 | 259586 |
| chr2 | 201284960 | 201284965 | 0.09 | 189088 | 0.27 | 124572 | 0.90 | 247499 |
| chr4 | 186617917 | 186617917 | 0.32 | 130816 | 0.45 | 77903 | 1.53 | 161881 |
| chr9 | 21971048 | 21971059 | 0.11 | 121096 | 0.24 | 77083 | 0.27 | 166840 |

Testing negative controls (saliva from healthy individuals): To ascertain the specificity of the assay, genomic DNA extracted from the saliva/oral rinse of 36 healthy individuals with no history of cancer as negative controls was sequenced. All 36 samples had generated >10M reads with >50000X average coverage. The read quality-reflected by the (230 scores was >90% and an alignment of >99% to the reference human genome with >95% on target reads were observed in all samples. At 0.3% VAF, 34 of the 36 samples had no reportable variants identified, generating a specificity of 94.4%. As expected, all samples (100%) were negative for HPV16/18 DNA,

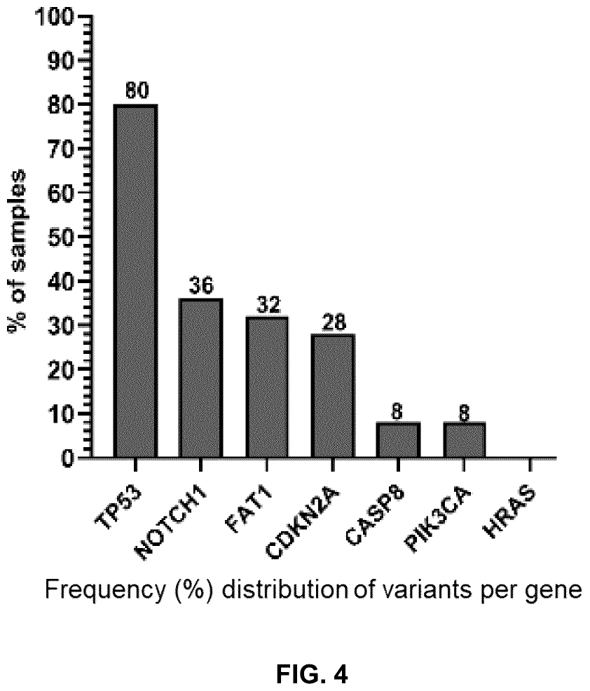

Premalignant FFPE samples: 32 premalignant (oral dysplasia) FFPE samples were sequenced and variant calling was done at 4% (standard cutoff for tissue samples). Variants were identified in 25/32 premalignant FFPE samples. The most frequently mutated gene was TP53, followed by NOTCH1 and FAT1 genes. The summary of mutations in each gene across 25 samples is shown in FIG. 4. All the FFPE samples were HPV negative, with alignment of less than 0.01% with to the HPV genome.

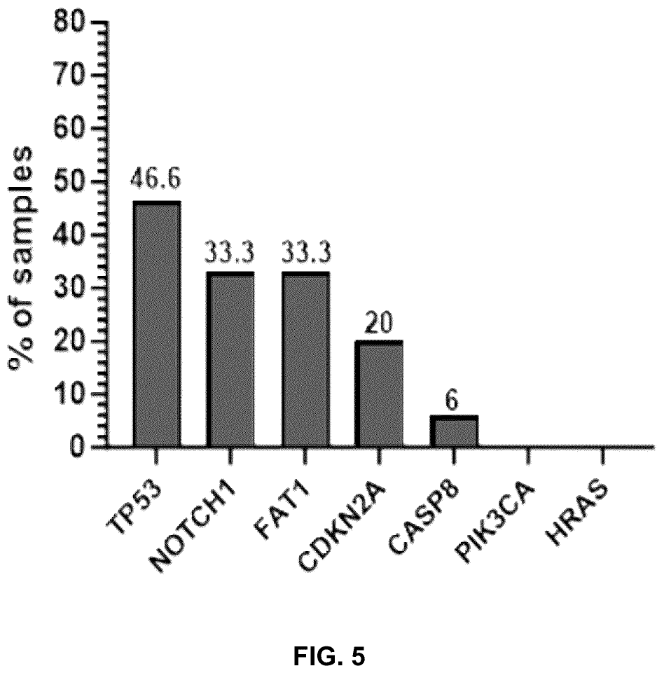

Premalignant saliva samples: 22 saliva samples collected from patients with oral dysplastic lesions were sequenced and variants were called at 0.2% VAF (accepted cutoff for saliva samples). Mutations were identified in 15 of 22 (68.2%) saliva samples. The summary of mutations in each gene across 15 samples is shown in FIG. 5. All the saliva samples (100%) were HPV negative.

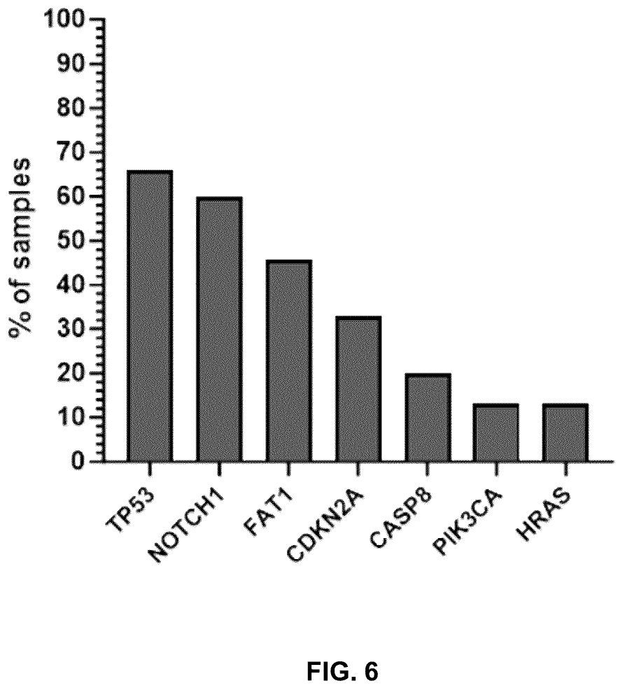

OCSCC FFPE samples: The dual assay was tested for mutation detection in 20 FFPE samples collected from OCSCC patients. Mutation variants were detected in all (100%) specimens at 4% VAF. The mutational frequencies among the 20 samples resembled the mutation patterns seen in the TCGA-HNSC dataset. All patients were HPV negative using a 5% alignment cutoff. The summary of variants in each gene across all 15 samples are summarized in FIG. 6.

HPV detection in OPSCC FFPE samples: To test the assay for HPV detection, 15 FFPE samples from HPV driven oropharyngeal squamous cell carcinoma (OPSCC) were sequenced. The assay was able to detect HPV in 13/15 samples at a 5% alignment cutoff. The results were validated through clinical HPV PCR assay performed in the CLIA certified lab. Interestingly, one of the two samples that tested HPV negative on our assay turned out to be HPV33 positive when tested with CLIA certified clinical HPV PCR assay. This could explain the negative result with our assay, which is designed to detect the most prevalent high-risk HPV strains (16 & 18). Mutations in TP53m PIK3CA, and FAT1 were detected in 12 of 15 cases.

HPV & mutation detection in OPSCC saliva samples: Further tested were ab additional 14 saliva samples from OPSCC patients for HPV and mutations in the 7 gene panel. Twelve of 14 patients were HPV positive and 8/14 had mutations in at least one of the 7 genes tested. Generally, HPV-negative HNSCCs are significantly more mutated than HPV-positive HNSCC.

HPV & Mutation detection in OCSCC Saliva Samples (ACS Cohort): From the ACS cohort, 233/543 samples have been sequenced. Those 233 samples include 81 cases and 152 controls. All the cases and controls (100%) were negative for HPV DNA.

1. A method of detecting a head and neck cancer (HNC), the method comprising performing an assay on an oral sample from a subject to detect: (i) a DNA sequence of NOTCH1, TP53, FAT1, PIK3CA, CDKN2A, HRAS, or CASP8; and (ii) the presence or absence of human papilloma virus (HPV).

2. The method of claim 1, wherein the HNC is a squamous cell carcinoma.

3. The method of claim 2, wherein the HNC is oropharngyl squamous cell carcinoma (OPSCC).

4. The method of claim 2, wherein the HNC is oral cavity squamous cell carcinoma (OCSCC).

5. The method of claim 4, wherein the method detects more than one DNA sequence of NOTCH1, TP53, FAT1, PIK3CA, CDKN2A, HRAS, or CASP8.

6. The method of claim 1, wherein the method detects the presence of absence of HPV 16 or HPV 18.

7. The method of claim 1, wherein the oral sample comprises saliva from the subject.

8. The method of claim 1, wherein the oral sample is an oral rinse sample from the subject.

9. The method of claim 1, wherein the oral sample comprises genetic material from the subject's oral cavity.

10. The method of claim 1, wherein the detecting the DNA sequence comprises sequencing DNA in the oral sample.

11. The method of claim 10, wherein sequencing DNA in the oral sample comprises next generation sequencing (NGS).

12. The method of claim 1, wherein the detecting the DNA sequence comprises targeted sequencing of the NOTCH1, TP53, FAT1, PIK3CA, CDKN2A, HRAS, and/or CASP8 genes.

13. The method of claim 1, wherein detecting the DNA sequence identifies the subject as having cancer.

14. The method of claim 1, wherein the detecting the presence or absence of HPV comprises sequencing.

15. The method of claim 1, wherein detecting the presence or absence of HPV determines a type of HNC.

16. The method of claim 15, wherein the presence of HPV indicates that the subject has or is at high risk of having OPSCC.

17. The method of claim 15, wherein the absence of HPV indicates that the subject has or is at high risk of having OCSCC.

18. A method of detecting oral HPV, the method comprising performing an assay of an oral sample to detect the presence or absence of oral HPV.

19. The method of claim 18, wherein the oral HPV is HPV 16 or HPV 18.

20. A kit comprising: one or more reagents that detect from an oral sample: (i) DNA comprising a sequence from a gene selected from NOTCH1, TP53, FAT1, PIK3CA, CDKN2A, HRAS, and/or CASP8 gene; and (ii) the presence or absence of human papilloma virus (HPV).
